(19) United States
(12) Patent Application Publication
(10) Pub. No.: US 2016/0215016 A1
(43) Pub. Date: Jul. 28, 2016

(54) SYNTHESIS OF PEPTIDE EPOXY KETONES

(71) Applicant: SANDOZ AG, Basel (CH)

(72) Inventors: Kathrin HÖFERL-PRANTZ, Kundl (AT); Thorsten WILHELM, Langkampfen (AT)

(21) Appl. No.: 14/915,580

(22) PCT Filed: Aug. 20, 2014
(86) PCT No.: PCT/EP2014/067727

§ 371 Date: Feb. 29, 2016

(30) Foreign Application Priority Data
Sep. 6, 2013 (EP) 13183304.8

(51) Int. Cl. C07K 5/107; C07C 225/06; C07C 53/18; C07D 301/12

(52) U.S. Cl. CPC C07K 5/1016; C07D 301/12; C07C 225/06; C07C 53/18

(57) ABSTRACT

It is provided an improved process for preparing peptide epoxy ketones, including novel compounds that can be used as intermediates in the process for preparing Carfilzomib and other peptide epoxy ketones.

Peptide epoxy ketones are an important class of proteasome inhibitors. One example is Carfilzomib. It is a tetrapeptide epoxy ketone and a selective proteasome inhibitor. It is an analog of epoxomicin.

The US FDA approved it for relapsed and refractory multiple myeloma. It is marketed under the trade name Kyprolis®.

The chemical name of Carfilzomib is (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide, represented by the following chemical structure:

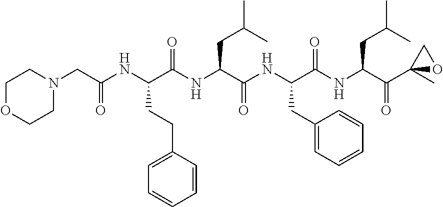 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

A specific route to Carfilzomib is described in WO2005105827 A2 and WO2006017842 A1. Both applications describe as a last step in the synthesis route the coupling of an epoxide of formula

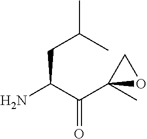

to a peptide precursor of formula

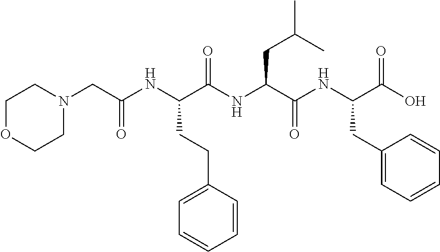 [SMILES: CC(C)C[C@H](NC(=O)[C@H](CCc1ccccc1)NC(=O)CN1CCOCC1)C(=O)N[C@@H](Cc1ccccc1)C(=O)O]

to obtain Carfilzomib. This way the stereocentre of the epoxide is formed in a small molecule. The epoxide is synthetised according to Crews, C. M. et al, Bioorg. Med. Chem. Letter 1999, 9, 2283-2288:

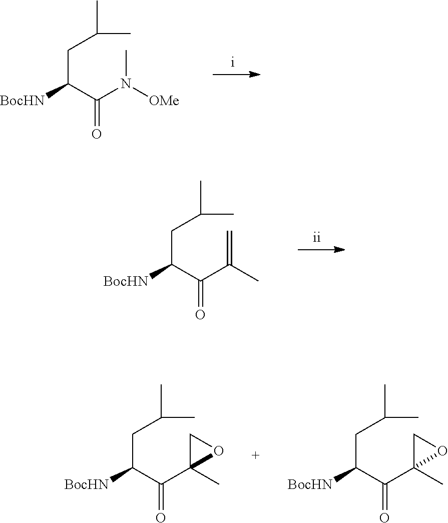

i) 2-Bromopropene, t-BuLi, Et₂O, −78° C., 2.5 h; ii) H₂O₂, H₂O, Benzonitrile, i-Pr₂EtN, MeOH, 0-4° C., 43 h, 1.7:1.

The Boc-protected vinyl ketone is epoxidized in one step with alkaline hydrogen peroxide, leading to a mixture of the diastereomers in a ratio of 1.7:1. The separated diastereomers were obtained after column chromatography.

WO2009045497 describes the same synthesis route to Carfilzomib as WO2005105827 A2 and WO2006017842. Differences are observed in the synthesis of the epoxide building block starting from the vinyl ketone. One route leads from the vinyl ketone over reduction, epoxidation and oxidation to the desired epoxide. This route is also disclosed in WO2005111009. A second route is a one step reaction from the vinyl ketone to the epoxide by an aqueous solution of NaOCl, leading however to a diastereomeric mixture which is purified by column chromatography.

All these synthesis routes leading to Carfilzomib, but also to peptide epoxy ketones in general, have the disadvantage that the epoxide is formed during the synthesis route as a building block and that the epoxide is not formed with high stereoselectivity. Hence, the yield of the epoxide building block with the desired configuration is very low. Further, the toxic epoxide building block is formed as an intermediate, which has to be handled over additional steps to obtain the final product.

Hence, it was an object of the present invention to overcome the above-mentioned disadvantages.

It was an object of the present invention to provide a process for preparing peptide epoxy ketones, especially Carfilzomib, with a high yield and/or a high grade of purity.

Further, the use of hazardous, expensive and dangerous substances should be avoided as much as possible.

Finally, it was an object of the invention to provide substances and/or a process assuring a straightforward reaction and preventing the formation of side products.

### SUMMARY OF THE INVENTION

It was found that the substances and/or method of the present invention could be used to improve the purity and the yield of process, such as the preparation of peptide epoxy ketones. Further advantages of the process of the invention are simple reaction conditions, the use of readily available starting materials and reagents, the use of solvents that are easy to handle and/or easily removed, the prevention of the use of hazardous and explosive materials and an improved stereoselectivity.

Thus, the above objectives are solved by the provision of an improved process for preparing peptide epoxy ketones, including novel compounds that can be used as intermediates in the process for preparing Carfilzomib and other peptide epoxy ketones.

### DETAILED DESCRIPTION OF THE INVENTION

In one embodiment of the invention, a method for preparing a compound of formula (I)

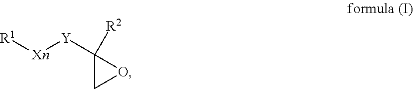

or a pharmaceutically acceptable salt or solvate thereof is described, wherein the method comprises:
(i) Providing a comp ound of formula (II)

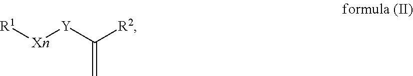

(ii) Epoxidizing the compound of formula (II) under conditions to obtain the compound of formula (I) or a pharmaceutically acceptable salt, hydrate or solvate thereof,
   wherein
   n is an integer between 1 and 1.000; 1 and 500; 1 and 200; 1 and 100; 1 and 50; 1 and 20; 1 and 10; preferably 1, 2, 3, 4, 5, 6, 7, 8, 9, 10; more preferably 2, 3, 4, 5, 6; most preferably 3,
   R¹ is R³-A-Q,
   Q is selected from C(O), C(S), C—OH, C—SH, SO₂; or Q is absent,
   A is selected from O, NH, C₁₋₇-alkyl, C₁₋₇-alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S; or A is absent,
   R³ is selected from PG (protecting group), (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, the heteroatom is selected from O, N and/or S, wherein in case of nitrogen it can be provided as N-Oxide,
   PG is a nitrogen-protecting group, preferably selected from carbamates, amides, N-alkyl and N-aryl amines, quaternary ammonium salts, N-sulfonyl derivatives, halogen, such as phthaloyl (Phth), tetrachlorophthaloyl (TCP), dithiasuccinyl (Dts), Trifluoroacetyl, methoxycarbonyl, ethoxycarbonyl, tert-Butoxycarbonyl (Boc), Benzyloxycarbonyl (Cbz), allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylsulfonyl (Ns), 2-(trimethylsilyl)ethylsulfonyl (SES), benzyl (Bn), diphenylmethyl (Dpm), p-methoxybenzyl (PMB), 3,4-dimethoxy benzyl (DMPM), p-methoxyphenyl (PMP) and allyl,
   R² is selected from hydrogen, linear or branched C₁₋₆-alkyl,
   Xn is a chain of amino acids of n units X, each unit X is NR⁴—CHR⁵—C(O), R⁴ and R⁵ of adjacent units X are independently equal or different, preferably R⁵ between adjacent units is different,
   Y is NR⁶—CHR⁷—C(O),
   each R⁴ and R⁶ are independently selected from hydrogen, linear or branched C₁₋₆-alkyl,
   each R⁵ and R⁷ are independently selected from hydrogen, linear or branched C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
(iii) optionally replacing the PG by another group as defined for R³, provided that R³ is selected from PG.

As used herein, linear or branched C₁₋₆-alkyl is selected from methyl, ethyl, n-propyl, isopropyl, n-butyl, isobutyl, sec-butyl, t-butyl, n-pentyl, isopentyl, t-pentyl, neo-pentyl, sec-pentyl, 3-pentyl, n-hexyl, sec-hexyl, t-hexyl, iso-hexyl.

In one embodiment of the method of the invention, n is 2, 3, 4, 5 or 6,

R² is selected from hydrogen, methyl, ethyl, n-propyl, isopropyl, isobutyl, sec-butyl, t-butyl, n-pentyl, isopentyl, t-pentyl, neo-pentyl, sec-pentyl, 3-pentyl, and
each R⁵ and R⁷ are independently selected from hydrogen, a naturally occurring amino acid side chain, a branched or unbranched aliphatic or aromatic group selected from ethyl, n-propyl, isopropyl, n-butyl, isobutyl, sec-butyl, t-butyl, aryl, benzyl, 1-phenylethyl, 2-phenylethyl, (1-naphthyl)methyl, (2-naphthyl)methyl, 1-(1-naphthyl)ethyl, 1-(2-naphthyl)ethyl, 2-(1-naphthyl)ethyl, 2-(2-naphthyl)ethyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S.

In an embodiment of the method of the invention,

R² is selected from hydrogen, methyl, ethyl, n-propyl, isopropyl,
R⁶ is hydrogen or C₁₋₆-alkyl,
R⁷ is selected from hydrogen, methyl, isopropyl, sec-butyl, isobutyl, homobenzyl or benzyl.

In a further embodiment of the method of the invention,

### Q is C(O),

A is C₁₋₇-alkyl,
R³ is selected from benzothienyl, naphthothienyl, thianthrenyl, furyl, pyranyl, isobenzofuranyl, chromenyl, pyrrolyl, imidazolyl, pyrazolyl, pyridyl, pyrazinyl, indolyl, purinyl, quinolyl, morpholino, pyrimidyl, pyrrolidyl.

It has been surprisingly found that the epoxide could be formed from the respective olefin with an increased stereoselectivity after the last coupling reaction. Further, as the epoxide could be formed eventually as a last step of the synthesis of peptide epoxy ketones, safety of the process could also be increased.

If the optional reaction step (iii) is a deprotection reaction, it can be carried out in an organic solvent or in a mixture of an organic solvent and water. Examples of organic solvents are dichloromethane, ethylacetate and alcohol such as methanol, ethanol and propanol, preferably ethanol. A mixture of ethanol and water is especially preferred. In one embodiment, deprotection can be carried out under acidic conditions, for example through the addition of a strong acid, such as hydrochloric acid, trifluoroacetic acid, sulphuric acid, nitric acid or an acidic cation exchanger, such as Amberlite IR 120 H⁺, preferably by the addition of hydrochloric acid or trifluoroacetic acid. In another embodiment, the deprotection can be carried out under basic conditions, for example through the addition of an anorganic base, such as sodium hydroxide, lithium hydroxide, potassium hydroxide or carbonate, sodium hydride or carbonate, or an organic base, such as triethyl amine, piperidine, morpholine or pyridine. In a further embodiment, cleavage of PG can be carried out under reductive conditions, such as with sodium borohydride, lithium aluminium hydride, zinc/acetic anhydride, sodium in liquid ammonia. In yet a further embodiment, the deprotection is carried out under oxidative conditions, such as with cerium ammonium nitrate (CAN) or 2,3-Dichloro-5,6-Dicyanobenzoquinone (DDQ). In one embodiment, the deprotection is carried out under hydrogenating conditions, such as with H₂/Pd/C or H₂/Pd black.

In a further embodiment of the method of the invention, Xn is selected from

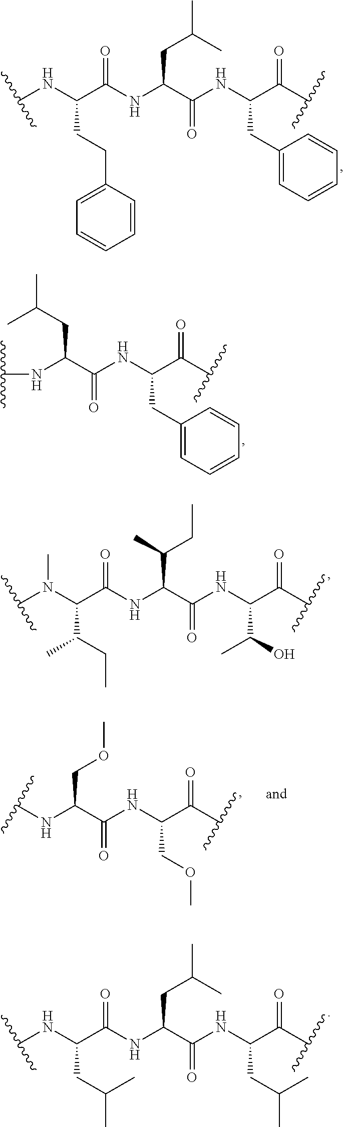

In a further embodiment of the method of the invention, Xn is represented by the formula

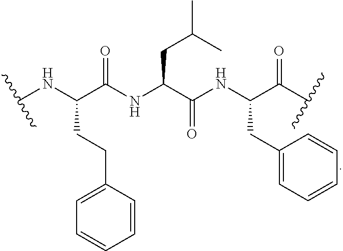 [SMILES: *N[C@@H](CCc1ccccc1)C(=O)N[C@@H](CC(C)C)C(=O)N[C@@H](Cc1ccccc1)C(*)=O]

In one embodiment of the method of the invention, the compound of formula (I) is selected from

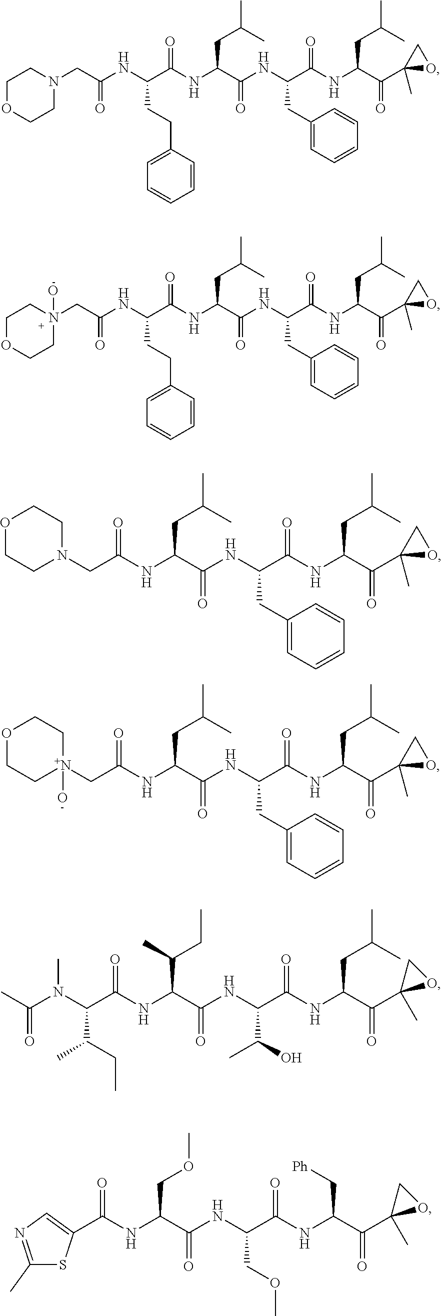

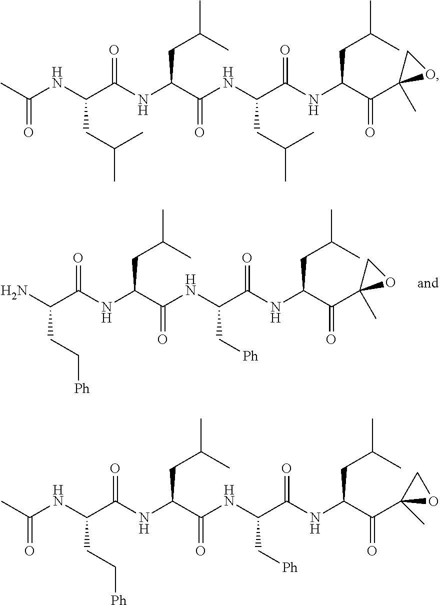 [SMILES: CC(=O)N[C@@H](CC(C)C)C(=O)N[C@@H](CC(C)C)C(=O)N[C@@H](CC(C)C)C(=O)N[C@@H](CC(C)C)C(=O)[C@@]1(C)CO1 ; CC(C)C[C@H](NC(=O)[C@@H](N)CCc1ccccc1)C(=O)N[C@@H](Cc1ccccc1)C(=O)N[C@@H](CC(C)C)C(=O)[C@@]1(C)CO1 ; CC(=O)N[C@@H](CCc1ccccc1)C(=O)N[C@@H](CC(C)C)C(=O)N[C@@H](Cc1ccccc1)C(=O)N[C@@H](CC(C)C)C(=O)[C@@]1(C)CO1]

All compounds of formula (I) are physiologically active as proteasome inhibitors.

Preferably, the compound of formula (I) is

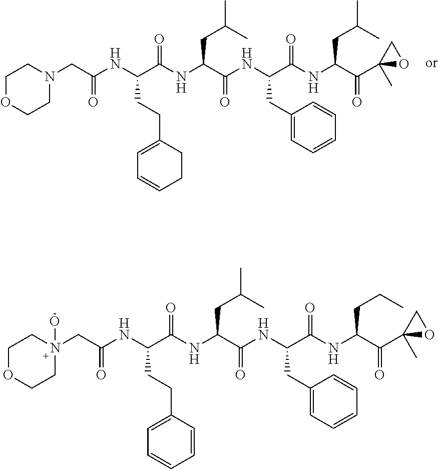

In an embodiment of the invention, the epoxidation step (ii) is performed subsequent to reaction step (iii).

In a second embodiment of the invention, the epoxidation step (ii) is performed prior to reaction step (iii).

In a further embodiment of the method, the epoxidation step (ii) is the final reaction step or the penultimate reaction step prior to performing reaction step (iii), reaction step (iii) being the final step.

In an embodiment of the method of the invention, the epoxidation step (ii) comprises subjecting the compound of formula (II) to an epoxidizing agent, wherein the epoxidizing agent is selected from hydrogen peroxide, organic peroxides like tert-butyl hydroperoxide, preferably peracids such as chloroperbenzoic acid, peracetic acid, more preferably chloroperbenzoic acid, anorganic peroxides, preferably hypochlorites, or a combination thereof, under conditions to obtain a compound of formula (I).

The epoxidizing agent is preferably hydrogen peroxide and the epoxidation step (ii) comprises subjecting the compound of formula (II) to an aqueous hydrogen peroxide solution under conditions that allow conversion to a compound of formula (I).

In one embodiment, the epoxidation reaction of step (ii) is carried out in an organic solvent, such as methanol, dichloromethane, N-methylpyrrolidone, acetonitrile, dimethyl formamide, preferably methanol or dichloromethane. The reaction can be carried out at a temperature in a range between −15-10° C., preferably −10-5° C., more preferably −5-3° C. With hydrogen peroxide as epoxidizing agent, the reaction is carried out in the presence of a hydroxide, such as potassium hydroxide or sodium hydroxide.

The use of hydrogen peroxide in combination with an inorganic hydroxide, such as potassium hydroxide or sodium hydroxide, provides epoxides with higher stereoselectivity compared to other epoxidizing agents.

According to an embodiment of the invention, the compound of formula (II) is prepared by a process comprising the steps:

Reacting a compound of formula (III)

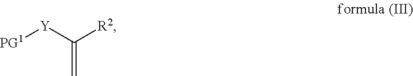

or a compound of formula (IV)

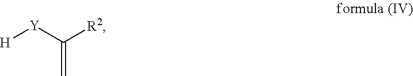

or a salt of a compound of formula (IV) with a compound of formula (V)

H¹-Xn-OH formula (V),

under conditions to obtain the compound of formula (II),

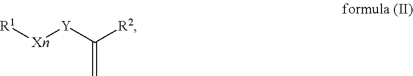

wherein
n, R¹, R², Xn and Y are defined as above, PG¹ is as defined as PG.

In a second embodiment of the invention, the compound of formula (II) is prepared by a process comprising the steps:

Reacting a compound of formula (III)

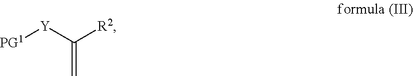

or a compound of formula (IV)

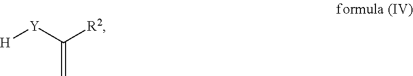

or a salt of a compound of formula (IV) with a compound of formula (VI)

PG²-X(n-m)-OH formula (VI),

under conditions to obtain the compound of formula (VII),

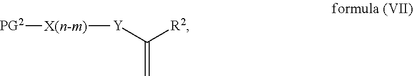

and subsequently coupling of m units X sequence wise, or of a sequence of m units X, according to the sequence Xn, with the compound of formula (VII) to obtain the compound of formula (II),

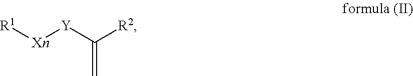

wherein
R¹, PG¹, R², X and Y are defined as above,
PG² is a nitrogen-protecting group, preferably selected from carbamates, amides, N-alkyl and N-aryl amines, quaternary ammonium salts, N-sulfonyl derivatives, halogen, such as phthaloyl (Phth), tetrachlorophthaloyl (TCP), dithiasuccinyl (Dts), Trifluoroacetyl, methoxycarbonyl, ethoxycarbonyl, tert-Butoxycarbonyl (Boc), Benzyloxycarbonyl (Cbz), allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylsulfonyl (Ns), 2-(trimethylsilyl)ethylsulfonyl (SES), benzyl (Bn), diphenylmethyl (Dpm), p-methoxybenzyl (PMB), 3,4-dimethoxy benzyl (DMPM), p-methoxyphenyl (PMP) and allyl,
n is an integer between 2 and 1.000; preferably 2, 3, 4, 5, 6, 7, 8, 9, or 10,
m is an integer between 1 and n−1,
X(n−m) is a chain of amino acids of n units X of sequence Xn, lacking an amino (N—) terminal sequence of m units X of the sequence Xn, each unit X is NR⁴—CHR⁵—C(O), R⁴ and R⁵ of adjacent units X are independently equal or different, preferably R⁵ between adjacent units is different, wherein R⁴ and R⁵ are defined as above.

The reaction of compounds of formula (III) or (IV) with a compound of formula (V) leading to the compound of formula (II) as well as the reaction to the compound of formula (VII) are peptide bond forming reactions. The peptide bond formation can be carried out according to known procedures. In one embodiment, the carboxy function of the compound of formula (V) is activated by a coupling agent such as a carbodiimide and/or a triazol. Examples of coupling agents are DCC (dicyclohexylcarbodiimide), DIC (diisopropylcarbodiimide), HOBt (1-hydroxy-benzotriazole), HOAt (1-hydroxy-7-aza-benzotriazole), BOP (benzotriazol-1-yloxy)tris(dimethylamio)phosphonium hexafluorophosphate), PyBOP (benzotriazol-1-yloxy)tris(pyrrolidino)phosphonium hexafluorophosphat, PyBroP (bromo)tris(pyrrolidino)phosphonium hexafluorophosphate), BroP (bromo)tris(dimethylamio)phosphonium hexafluorophosphate), HBTU (2-(1H-benzotriazole-1-yl)-1,1,3,3-tetramethyluronium hexafluorophosphate) and mixtures thereof. Additionally, it is preferred that an organic alkaline substance, preferably an amine, is present in the mixture. Examples of the organic alkaline substance are triethylamine and DIPEA (diisopropylethylamin), in particular DIPEA. The reaction can be carried out in an organic solvent, such as dimethyl formamide (DMF), dimethylsulfoxide, DMPU, acetonitrile and dichloromethane, preferably DMF. In one embodiment, the solvent is a mixture of at least two organic solvents, such as DCM/DMF.

In one embodiment of the invention, the peptide bond forming reactions to obtain the compound of formula (II) and/or formula (VII) are performed in the presence of at least one Lewis acid, preferably CuCl₂. The use of CuCl₂ reduces the risk of epimerization during the peptide bond formation, thus enabling the development of convergent synthesis routes.

In one embodiment of the method of the invention, the compound of formula (II) is prepared by a process comprising the steps:

Reacting a compound of formula (III)

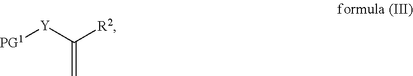

or a compound of formula (IV)

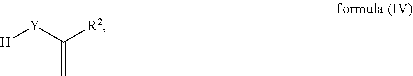

or a salt of a compound of formula (IV) with a compound of formula (V) selected from

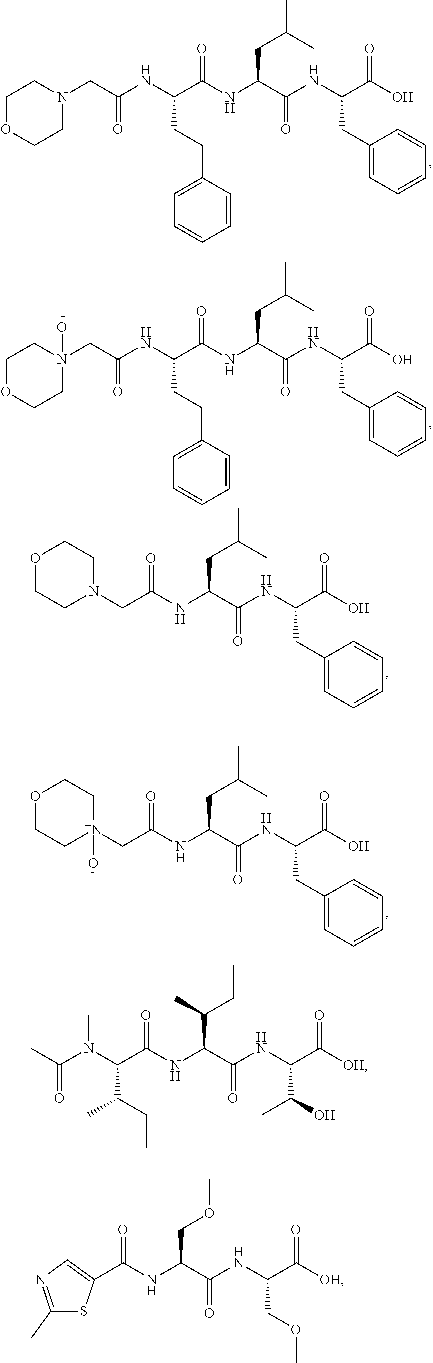

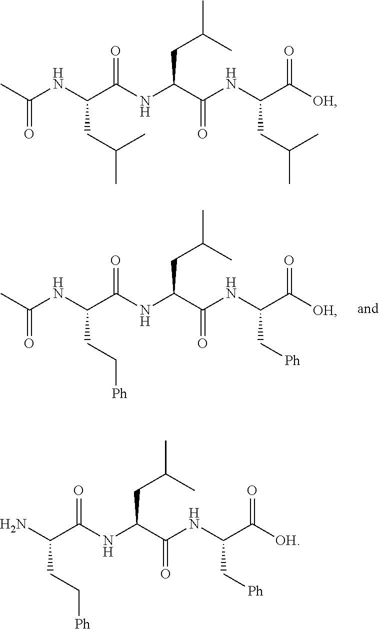

This is an example of a convergent synthesis route, as the peptide precursor of formula (V) and the compound of formula (III) or (IV) are synthesized separately and coupled at a late stage. As described above, the reaction between the two compounds is preferably carried out in the presence of a Lewis acid, preferably CuCl₂.

In an embodiment of the method of the invention, the preparation of the compound of formula (II) comprises the steps:

Reacting a compound of formula (III)

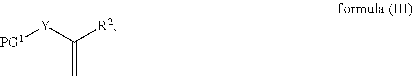

or a compound of formula (IV)

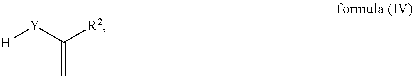

or a salt of a compound of formula (IV) with a compound of formula (VIII)

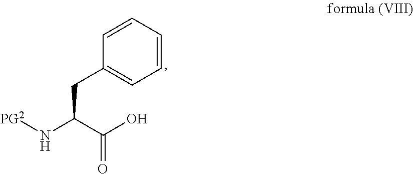

under conditions to obtain the compound of formula (IX),

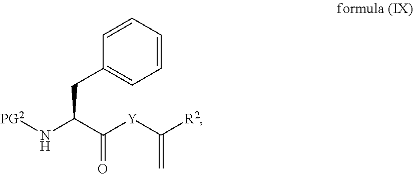

Reacting the compound of formula (IX) with a compound of formula (X)

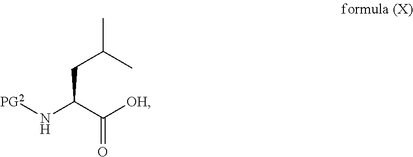

under conditions to obtain the compound of formula (XI),

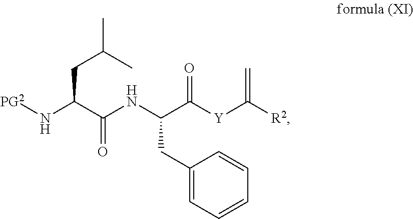

Reacting the compound of formula (XI) with a compound of formula (XII)

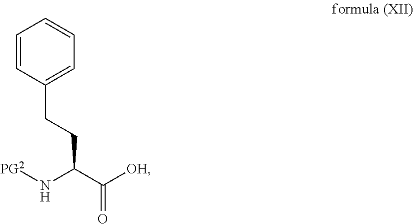

or with a compound of formula (XIII)

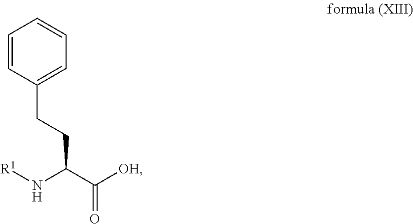

under conditions to obtain the compound of formula (XIV) or (XV),

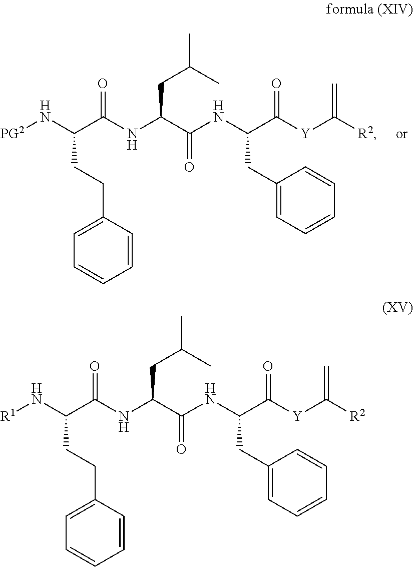

optionally, subsequently replacing the structural component PG² of the compound of formula (XIV) by the structural component R¹,
wherein
R¹, PG¹, R², Y and PG² are defined as above.

Also the reactions leading to the compounds of formula (IX), (XI), (XIV) and (XV) are peptide bond formation reactions. The peptide bond formation can be carried out according to known procedures. In one embodiment, the carboxy function of the compounds of formula (VIII), (X) and (XII) is activated by a coupling agent such as a carbodiimide and/or a triazol. Examples of coupling agents are DCC (dicyclohexylcarbodiimide), DIC (diisopropylcarbodiimide), HOBt (1-hydroxy-benzotriazole), HOAt (1-hydroxy-7-aza-benzotriazole), BOP (benzotriazol-1-yloxy)tris(dimethylamio)phosphonium hexafluorophosphate), PyBOP (benzotriazol-1-yloxy)tris(pyrrolidino)phosphonium hexafluorophosphat, PyBroP (bromo)tris(pyrrolidino)phosphonium hexafluorophosphate), BroP (bromo)tris(dimethylamio)phosphonium hexafluorophosphate), HBTU (2-(1H-benzotriazole-1-yl)-1,1,3,3-tetramethyluronium hexafluorophosphate) and mixtures thereof. Additionally it is preferred that an organic alkaline substance, preferably an amine, is present in the mixture. Examples of the organic alkaline substance are triethylamine and DIPEA (diisopropylethylamine), in particular DIPEA. The reaction can be carried out in an organic solvent, such as DMF, dimethylsulfoxide, DMPU, acetonitrile and DCM, preferably DMF. In one embodiment, the solvent is a mixture of at least two organic solvents, such as DCM/DMF.

In one embodiment of the invention, the peptide bond forming reactions to obtain the compound of formula (IX) and/or formula (XI) and/or formula (XIV) or (XV) as described above, are performed in the presence of at least one Lewis acid, preferably CuCl₂. The use of CuCl₂ reduces the risk of epimerization during the peptide bond formation, thus enabling the development of convergent synthesis routes.

In a further embodiment of the method of the invention, the compound of formula (II) is selected from

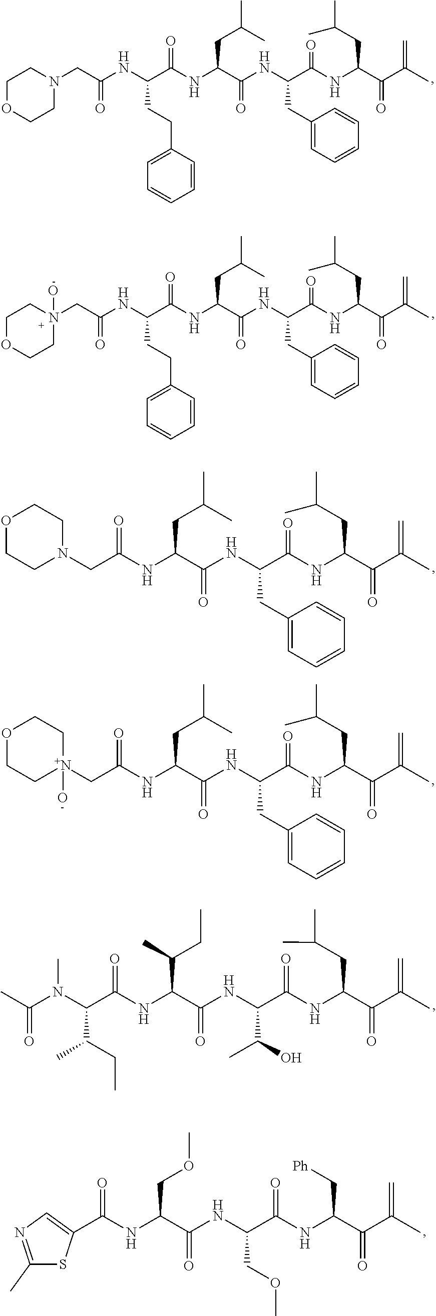

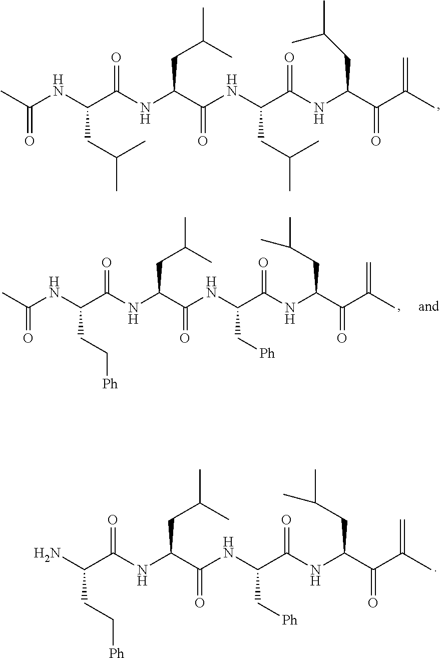

In a further embodiment of the invention, the compound of formula (III)

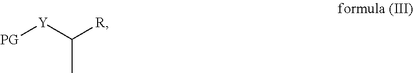

or the compound of formula (IV)

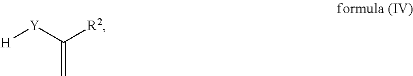

or a salt thereof
is prepared by a process comprising the steps of:
(a) Providing a compound of formula (XVI)

PG¹-Y-PG³ formula (XVI),
(b) Reacting the compound of formula (XVI) with a compound of formula (XVIa)

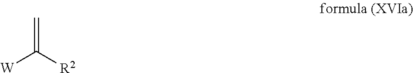

under conditions to obtain the compound of formula (III)
(c) Optionally removing of PG¹ of the compound of formula (III) under conditions to obtain the compound of formula (IV) or a salt thereof,
   wherein
   PG¹, R² and Y are defined as above.

PG³ is a Carboxyl-protection group, preferably a secondary amine, preferably selected from N,O-dimethylhydroxylamine, pyrrolidine or morpholine, preferably pyrrolidine.

W is selected from Li and MgHal (Grignard reagent), i.e. MgF, MgCl, MgBr and MgI. Preferably, W is MgHal, as the reaction with a Grignard reagent leads to the compound of formula (III) with a higher yield. Preferably, MgHal is MgBr.

The reaction between the compound of formula (XVI) and the compound of formula (XVIa) is carried out in an organic solvent such as diethylether and THF, preferably THF. Preferably, the Grignard reagent is added to the compound of formula (XVI) at room temperature, followed by stirring the resulting mixture at a temperature in the range of 40 to 60° C. Stirring the mixture at a temperature in the range of 40 to 60° C. increases the yield compared to stirring the reaction at room temperature or at 0° C.

Reaction step (c) leading to the compound of formula (IV) or a salt thereof can be carried out in an organic solvent or in a mixture of an organic solvent and water. Examples of organic solvents are dichloromethane, ethylacetate and alcohol such as methanol, ethanol and propanol, preferably ethanol. A mixture of ethanol and water is especially preferred. In one embodiment, deprotection can be carried out under acidic conditions, for example through the addition of a strong acid, such as hydrochloric acid, trifluoroacetic acid, sulphuric acid, nitric acid or an acidic cation exchanger, such as Amberlite IR 120 H⁺, preferably by the addition of hydrochloric acid or trifluoroacetic acid. In another embodiment, the deprotection can be carried out under basic conditions, for example through the addition of an anorganic base, such as sodium hydroxide, lithium hydroxide, potassium hydroxide or carbonate, sodium hydride or carbonate, or an organic base, such as triethyl amine, piperidine, morpholine or pyridine. In a further embodiment, cleavage of PG¹ can be carried out under reductive conditions, such as with sodium borohydride, lithium aluminium hydride, zinc/acetic anhydride, sodium in liquid ammonia. In yet a further embodiment, the deprotection is carried out under oxidative conditions, such as with cerium ammonium nitrate (CAN) or 2,3-Dichloro-5,6-Dicyanobenzoquinone (DDQ). In one embodiment, the deprotection is carried out under hydrogenating conditions, such as with H₂/Pd/C or H₂/Pd black.

In one embodiment Y, which is defined as NR⁶—CHR⁷—C(O), is selected such that

R⁶ is hydrogen or C₁₋₆-alkyl,
R⁷ is selected from hydrogen, methyl, isopropyl, sec-butyl, isobutyl, homobenzyl or benzyl.

In one embodiment of the method of the invention, the salt of the compound of formula (IV) is formed by a cation which is

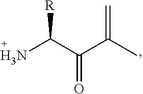

and an anion, the anion is preferably selected from F₃CCO₂⁻, nitrate, sulfate, halogen, such as chloride, bromide, iodide,
wherein
R⁷ is as defined above and preferably selected from hydrogen, methyl, isopropyl, sec-butyl, isobutyl, homobenzyl or benzyl.

In a further embodiment of the method of the invention the compound of formula (III) is

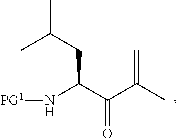

the compound of formula (IV) is

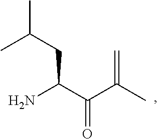

and/or the salt of the compound of formula (IV) is formed by a cation which is

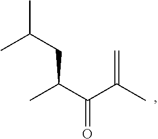

and an anion, the anion is preferably selected from F₃CCO₂⁻, nitrate, sulfate, halogen, such as chloride, bromide, iodide,
wherein PG¹ is a nitrogen-protecting group, preferably selected from carbamates, amides, N-alkyl and N-aryl amines, quaternary ammonium salts, N-sulfonyl derivatives, halogen, such as such as phthaloyl (Phth), tetrachlorophthaloyl (TCP), dithiasuccinyl (Dts), Trifluoroacetyl, methoxycarbonyl, ethoxycarbonyl, tert-Butoxycarbonyl (Boc), Benzyloxycarbonyl (Cbz), allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylsulfonyl (Ns), 2-(trimethylsilyl)ethylsulfonyl (SES), benzyl (Bn), diphenylmethyl (Dpm), p-methoxybenzyl (PMB), 3,4-dimethoxy benzyl (DMPM), p-methoxyphenyl (PMP) and allyl.

The present invention is also directed to a salt of the compound of formula (IV),

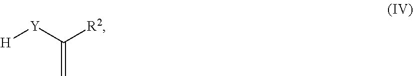

which is formed by a cation and an anion, the anion is preferably selected from F₃CCO₂⁻, nitrate, sulfate, halogen, such as chloride, bromide, iodide,
wherein
Y is NR⁶—CHR⁷—C(O),
R⁶ is selected from hydrogen, C₁₋₆-alkyl,
R⁷ is selected from C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
R² is selected from hydrogen, methyl, ethyl, n-propyl, isopropyl, isobutyl, sec-butyl, t-butyl, n-pentyl, isopentyl, t-pentyl, neo-pentyl, sec-pentyl, 3-pentyl, n-hexyl, sec-hexyl, t-hexyl, iso-hexyl.

Preferably, the salt of the compound of formula (IV) is formed by a cation which is

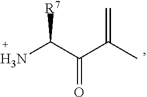

and an anion, the anion is preferably selected from halogen, F₃CCO₂⁻, nitrate, sulfate
wherein
R₇ is as defined above and preferably selected from hydrogen, methyl, isopropyl, sec-butyl, isobutyl, homobenzyl or benzyl
or
by a cation that is

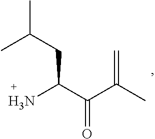

and an anion, the anion is preferably selected from F₃CCO₂⁻, nitrate, sulfate, halogen such as chloride, bromide, iodide.

In one embodiment, the salt of the compound of formula (IV) can be obtained by a method as disclosed above.

The present invention is also directed to novel compounds of formula (I)

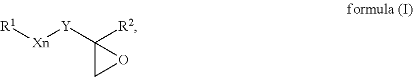

wherein n is 1, 2, 3, 4, 5 or 6, preferably 3,
R¹ is R³-A-Q,
Q is selected from C(O), C(S), C—OH, C—SH, SO₂; or Q is absent,
A is selected from O, NH, C₁₋₇-alkyl, C₁₋₇-alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S; or A is absent,
R³ is selected from PG (protecting group), hydrogen, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, the heteroatom is selected from O, N and/or S, wherein in case of nitrogen it can be provided as N-Oxide,
PG is a nitrogen-protecting group, preferably selected from carbamates, amides, N-alkyl and N-aryl amines, quaternary ammonium salts, N-sulfonyl derivatives, halogen such as such as phthaloyl (Phth), tetrachlorophthaloyl (TCP), dithiasuccinyl (Dts), Trifluoroacetyl, methoxycarbonyl, ethoxycarbonyl, tert-Butoxycarbonyl (Boc), Benzyloxycarbonyl (Cbz), allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylsulfonyl (Ns), 2-(trimethylsilyl)ethylsulfonyl (SES), benzyl (Bn), diphenylmethyl (Dpm), p-methoxybenzyl (PMB), 3,4-dimethoxy benzyl (DMPM), p-methoxyphenyl (PMP) and allyl,
R² is selected from hydrogen, linear or branched C₁₋₆-alkyl,
Xn is a chain of amino acids of n units X, each unit X is NR⁴—CHR⁵—C(O), R⁴ and R⁵ of adjacent units X are independently equal or different, preferably R⁵ between adjacent units is different;
Y is NR⁶—CHR⁷—C(O),
each R⁴ and R⁶ are independently selected from hydrogen, linear or branched C₁₋₆-alkyl,
each R⁵ and R⁷ are independently selected from hydrogen, C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,

In a further embodiment,

n is 2, 3, 4, 5 or 6, preferably 3,
each R⁵ and R⁷ are independently selected from hydrogen, a naturally occurring amino acid side chain, a branched or unbranched aliphatic or aromatic group selected from ethyl, n-propyl, isopropyl, n-butyl, isobutyl, t-butyl, aryl, 1-phenylethyl, 2-phenylethyl, (1-naphthyl)methyl, (2-naphthyl)methyl, 1-(1-naphthyl)ethyl, 1-(2-naphthyl)ethyl, 2-(1-naphthyl)ethyl, 2-(2-naphthyl)ethyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S.

In yet a further embodiment,

R⁶ is hydrogen or C₁₋₆-alkyl,
R⁷ is selected from hydrogen, methyl, isopropyl, sec-butyl, isobutyl, homobenzyl or benzyl.

In an embodiment of the invention,

### Q is C(O),

A is C₁₋₇-alkyl,
R³ is PG or benzothienyl, naphthothienyl, thianthrenyl, furyl, pyranyl, isobenzofuranyl, chromenyl, pyrrolyl, imidazolyl, pyrazolyl, pyridyl, pyrazinyl, indolyl, purinyl, quinolyl, morpholino, pyrimidyl, pyrrolidyl,
PG is a nitrogen-protecting group, preferably selected from carbamates, amides, N-alkyl and N-aryl amines, quaternary ammonium salts, N-sulfonyl derivatives, halogen such as such as phthaloyl (Phth), tetrachlorophthaloyl (TCP), dithiasuccinyl (Dts), Trifluoroacetyl, methoxycarbonyl, ethoxycarbonyl, tert-Butoxycarbonyl (Boc), Benzyloxycarbonyl (Cbz), allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylsulfonyl (Ns), 2-(trimethylsilyl)ethylsulfonyl (SES), benzyl (Bn), diphenylmethyl (Dpm), p-methoxybenzyl (PMB), 3,4-dimethoxy benzyl (DMPM), p-methoxyphenyl (PMP) and allyl.

In an embodiment of the invention,

Xn in the compound of formula (I) is an amino acid chain selected from

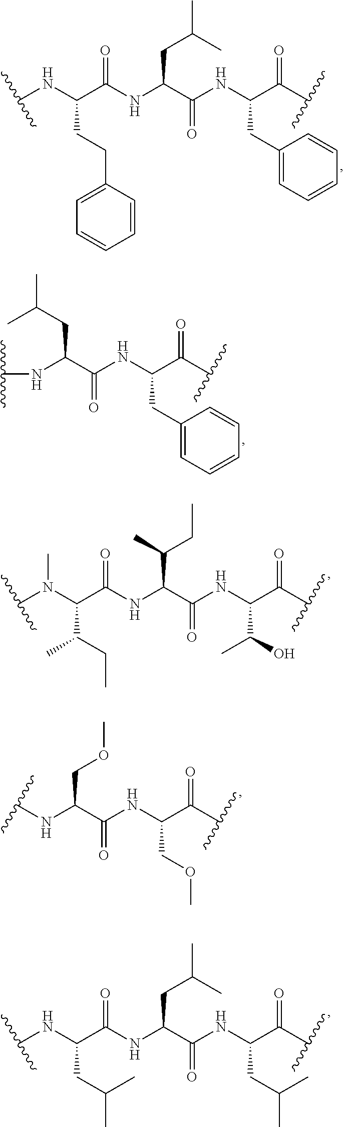

### or X is

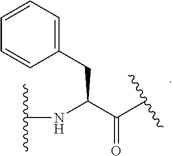 [SMILES: *N[C@@H](Cc1ccccc1)C(*)=O]

Preferably, Xn is represented by the formula

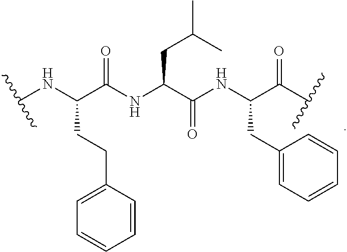 [SMILES: *N[C@@H](CCc1ccccc1)C(=O)N[C@@H](CC(C)C)C(=O)N[C@@H](Cc1ccccc1)C(*)=O]

In yet a further embodiment, the compound of formula (I) is selected from

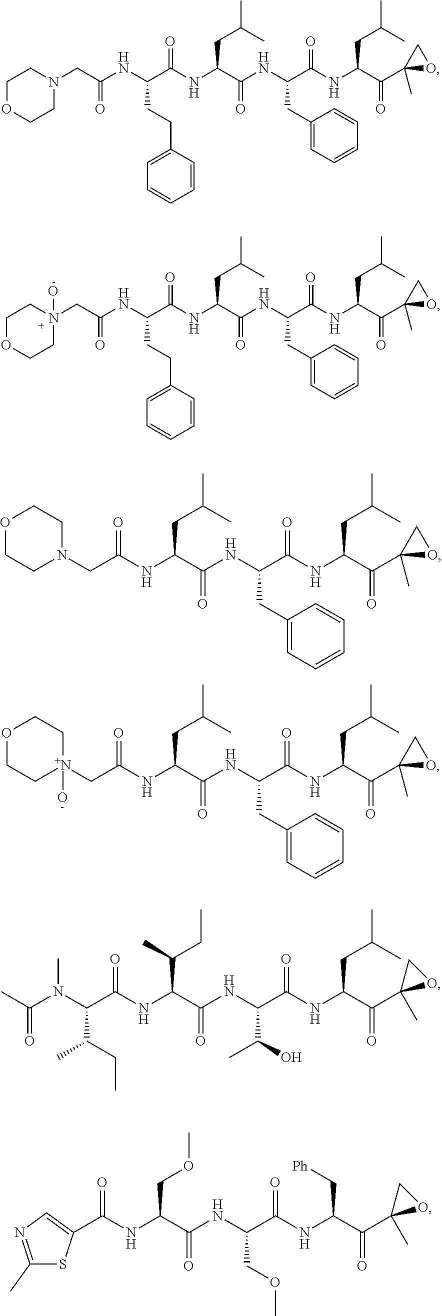

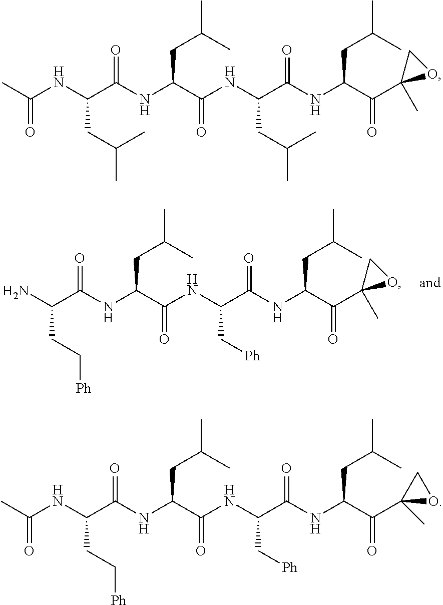 [SMILES: CC(=O)N[C@@H](CC(C)C)C(=O)N[C@@H](CC(C)C)C(=O)N[C@@H](CC(C)C)C(=O)N[C@@H](CC(C)C)C(=O)[C@@]1(C)CO1 ; CC(C)C[C@H](NC(=O)[C@@H](N)CCc1ccccc1)C(=O)N[C@@H](Cc1ccccc1)C(=O)N[C@@H](CC(C)C)C(=O)[C@@]1(C)CO1 ; CC(=O)N[C@@H](CCc1ccccc1)C(=O)N[C@@H](CC(C)C)C(=O)N[C@@H](Cc1ccccc1)C(=O)N[C@@H](CC(C)C)C(=O)[C@@]1(C)CO1]

Preferably, the compound of formula (I) is

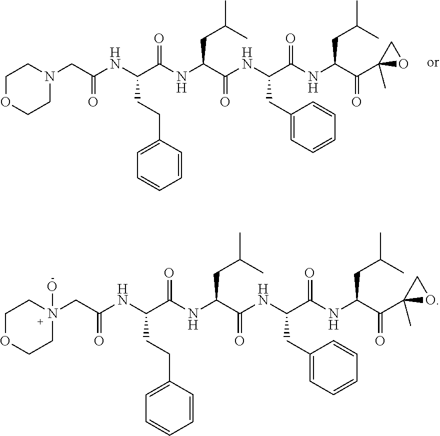

In one embodiment of the invention, the compound of formula (I) as defined in the specification is obtainable by any method as described herein.

In a further embodiment of the invention, the compound of formula (I) inhibits an enzymatic activity of an eukaryotic proteasome, when contacting said eukaryotic proteasome or a subunit thereof with the compound of formula (I) in vivo or in vitro.

The present invention is also directed to a compound of formula (II)

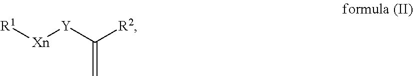

wherein
n is 2, 3, 4, 5, 6, 7, 8, 9 or 10; preferably 2, 3, 4, 5, 6; more preferably 3,
R¹ is R³-A-Q,
Q is selected from C(O), C(S), C—OH, C—SH, SO₂; or Q is absent,
A is selected from O, NH, C₁₋₇-alkyl, C₁₋₇-alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S; or A is absent,
R³ is selected from PG (protecting group), hydrogen, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, the heteroatom is selected from O, N and/or S, wherein in case of nitrogen it can be provided as N-Oxide,
R² is selected from linear or branched C₁₋₆-alkyl,
Xn is a chain of amino acids of n units X, each unit X is NR⁴—CHR⁵—C(O), R⁴ and R⁵ of adjacent units X are independently equal or different, preferably R⁵ between adjacent units is different;
Y is NR⁶—CHR⁷—C(O),
each R⁴ and R⁶ are independently selected from hydrogen, C₁₋₆-alkyl,
R⁵ is selected from hydrogen, C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
R⁷ is selected from hydrogen, C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S.

In a further embodiment,

n is 2, 3, 4, 5 or 6,
R⁵ is selected from hydrogen, a naturally occurring amino acid side chain, a branched or unbranched aliphatic or aromatic group selected from ethyl, n-propyl, isopropyl, n-butyl, isobutyl, t-butyl, aryl, 1-phenylethyl, 2-phenylethyl, (1-naphthyl)methyl, (2-naphthyl)methyl, 1-(1-naphthyl)ethyl, 1-(2-naphthyl)ethyl, 2-(1-naphthyl)ethyl, 2-(2-naphthyl)ethyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
R⁷ is selected from hydrogen, a naturally occurring amino acid side chain, a branched or unbranched aliphatic or aromatic group selected from ethyl, n-propyl, isopropyl, n-butyl, isobutyl, t-butyl, aryl, 1-phenylethyl, 2-phenylethyl, (1-naphthyl)methyl, (2-naphthyl)methyl, 1-(1-naphthyl)ethyl, 1-(2-naphthyl)ethyl, 2-(1-naphthyl)ethyl, 2-(2-naphthyl)ethyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S.

In yet a further embodiment,

R⁶ is hydrogen or C₁₋₆-alkyl,
R⁷ is selected from hydrogen, methyl, isopropyl, sec-butyl, isobutyl, homobenzyl or benzyl.

In further embodiment of the invention,

### Q is C(O),

A is C₁₋₇-alkyl,
R³ is PG or benzothienyl, naphthothienyl, thianthrenyl, furyl, pyranyl, isobenzofuranyl, chromenyl, pyrrolyl, imidazolyl, pyrazolyl, pyridyl, pyrazinyl, indolyl, purinyl, quinolyl, morpholino, pyrimidyl, pyrrolidyl,
PG is a nitrogen-protecting group, preferably selected from carbamates, amides, N-alkyl and N-aryl amines, quaternary ammonium salts, N-sulfonyl derivatives, halogen such as such as phthaloyl (Phth), tetrachlorophthaloyl (TCP), dithiasuccinyl (Dts), Trifluoroacetyl, methoxycarbonyl, ethoxycarbonyl, tert-Butoxycarbonyl (Boc), Benzyloxycarbonyl (Cbz), allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylsulfonyl (Ns), 2-(trimethylsilyl)ethylsulfonyl (SES), benzyl (Bn), diphenylmethyl (Dpm), p-methoxybenzyl (PMB), 3,4-dimethoxy benzyl (DMPM), p-methoxyphenyl (PMP) and allyl.

The compound of formula (II) is obtainable by any of the methods described herein.

In a further embodiment, Xn in the compound of formula (II) is represented by the formula

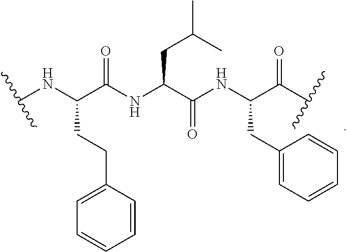 [SMILES: *N[C@@H](CCc1ccccc1)C(=O)N[C@@H](CC(C)C)C(=O)N[C@@H](Cc1ccccc1)C(*)=O]

In one embodiment of the invention, Xn of the compound of formula (II) is a sequence selected from

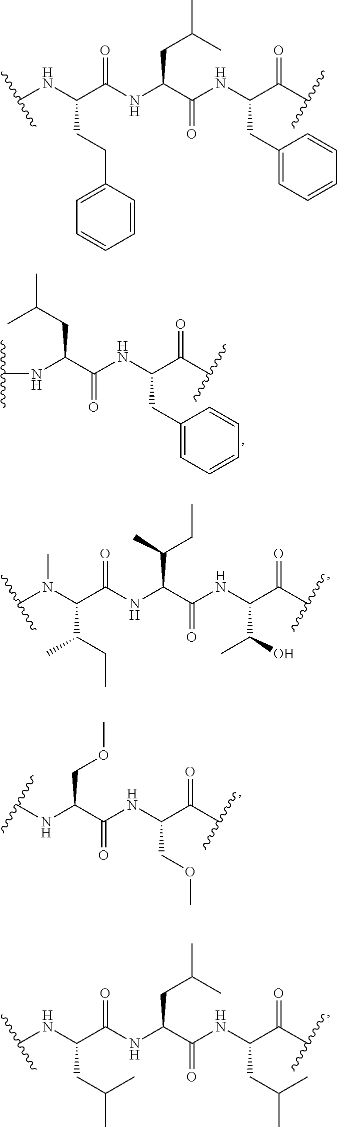

### or X is

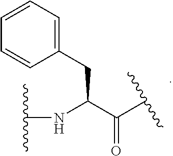 [SMILES: *N[C@@H](Cc1ccccc1)C(*)=O]

In a further embodiment of the invention the compound of formula (II) is selected from

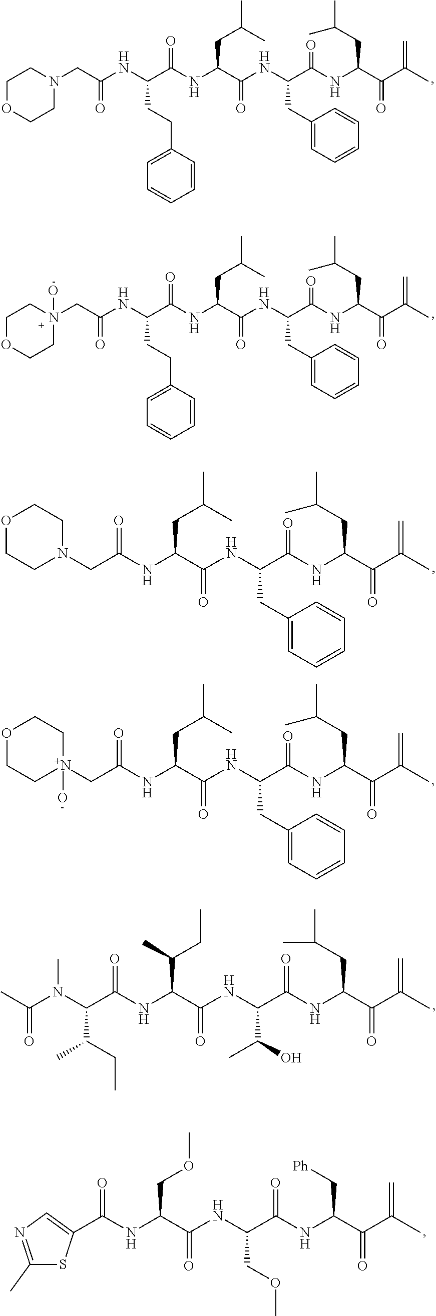

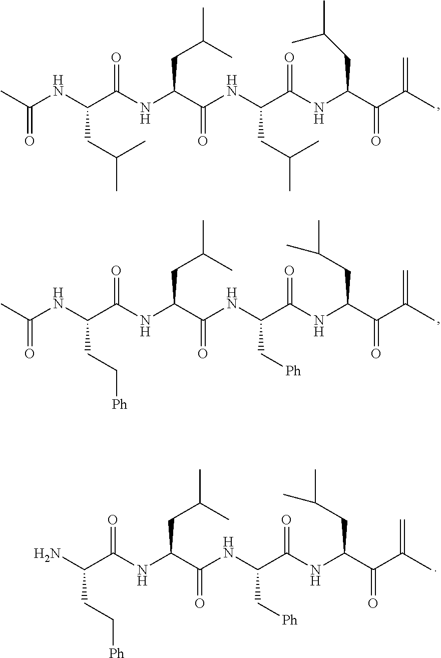

Preferably, the compound of formula (II) is

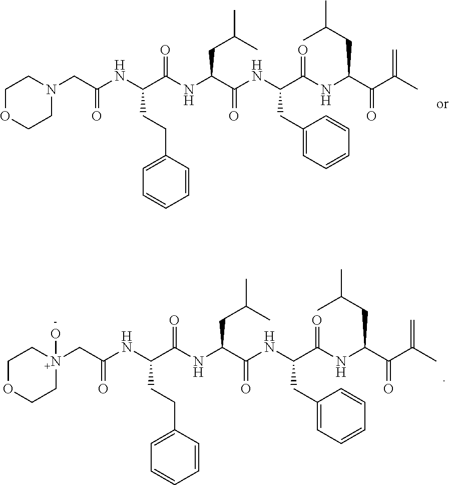 [SMILES: C=C(C)C(=O)[C@H](CC(C)C)NC(=O)[C@H](Cc1ccccc1)NC(=O)[C@H](CC(C)C)NC(=O)[C@H](CCc1ccccc1)NC(=O)CN1CCOCC1 ; C=C(C)C(=O)[C@H](CC(C)C)NC(=O)[C@H](Cc1ccccc1)NC(=O)[C@H](CC(C)C)NC(=O)[C@H](CCc1ccccc1)NC(=O)C[N+]1(O)CCOCC1]

The present invention is also directed to a composition, preferably a pharmaceutical composition, comprising a compound of formula (I) as defined above, wherein the composition is free or substantially free of a compound of formula (XVII)

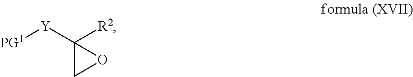

and/or formula (XVIII)

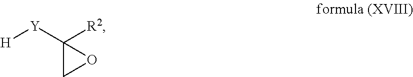

or a salt of the compound of formula (XVIII),
wherein the structural components Y and R² between the compounds of formulae (I), (XVII) and (XVIII) are identical, wherein
PG¹ is a nitrogen-protecting group, preferably selected from carbamates, amides, N-alkyl and N-aryl amines, quaternary ammonium salts, N-sulfonyl derivatives, halogen such as such as phthaloyl (Phth), tetrachlorophthaloyl (TCP), dithiasuccinyl (Dts), Trifluoroacetyl, methoxycarbonyl, ethoxycarbonyl, tert-Butoxycarbonyl (Boc), Benzyloxycarbonyl (Cbz), allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylsulfonyl (Ns), 2-(trimethylsilyl)ethylsulfonyl (SES), benzyl (Bn), diphenylmethyl (Dpm), p-methoxybenzyl (PMB), 3,4-dimethoxy benzyl (DMPM), p-methoxyphenyl (PMP) and allyl,
R² is selected from hydrogen, C₁₋₆-alkyl,
Y is NR⁶—CHR⁷—C(O),
R⁶ is selected from hydrogen, linear or branched C₁₋₆-alkyl, such as methyl, ethyl, propyl, isopropyl, sec-butyl, isobutyl, pentyl, hexyl,
R⁷ is selected from hydrogen, C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S.

The pharmaceutical composition as described above further contains between

0.005% (w/w) and 5% (w/w) or 0.01% (w/w) and 1% (w/w) of the compound of formula (II) as defined above, and/or
0.005% (w/w) and 5% (w/w) or 0.01% (w/w) and 1% (w/w) of the compound of formula (IV) as defined above, and
wherein the structural components Y and R² between the compounds of formulae (I), (II), (IV), (XVII) and (XVIII) are identical.

### EXAMPLES

### Example 1

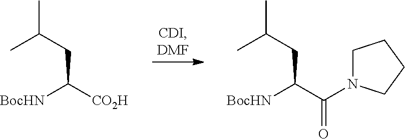 [SMILES: CC(C)C[C@H](NC(=O)OC(C)(C)C)C(=O)O ; CC(C)C[C@H](NC(=O)OC(C)(C)C)C(=O)N1CCCC1]

Boc-Leu-OH (47 g, 200 mmol) was dissolved in DMF (470 mL), CDI (36.8 g, 220 mmol) was added and stirred for 20 min. Pyrrolidine (18 mL, 220 mmol) was added slowly and the reaction was stirred at rt for 2 h. EtOAc (500 mL) and water (500 mL) were added to the reaction mixture. The layers were separated and the aqueous layer was extracted with EtOAc (500 mL). The combined organic layer was washed with 1N HCl (2×250 mL), 1N NaOH (2×250 mL) and water (4×250 mL), dried over MgSO₄ and solvent was removed under reduced pressure to give 47.8 g (90%) of the amide.

¹H NMR (500 MHz, CDCl₃) δ=5.22 (bd, J=9.60 Hz, 1H), 4.44 (dt, J=3.77, 9.69 Hz, 1H), 3.66 (dt, J=6.79, 9.78 Hz, 1H), 3.50 (dt, J=7.00, 12.10 Hz, 1H), 3.39 (dt, J=7.17, 10.90 Hz, 2H), 1.95 (m, 2H), 1.86 (m, 2H), 1.70 (m, 1H), 1.49 (ddd, J=4.34, 14.12, 9.97 Hz, 1H), 1.41 (s, 9H), 1.35 (ddd, J=4.25, 13.70, 9.30 Hz, 1H), 0.97 (d, J=6.65 Hz, 3H), 0.91 (d, J=6.60 Hz, 3H)

### Example 2

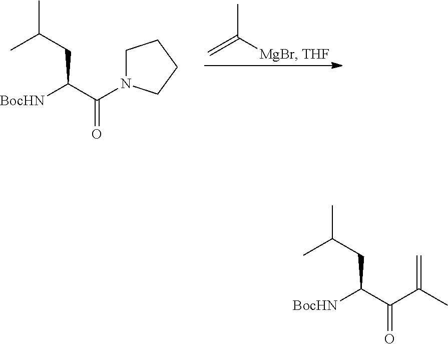

(S)-tert-butyl (4-methyl-1-oxo-1-(pyrrolidin-1-yl)pentan-2-yl)carbamate (10 g, 35.2 mmol) was dissolved in THF (30 mL) under N₂ at rt and the Grignard solution (176 mL, 88 mmol) was slowly added via dropping funnel. After the addition was finished, the reaction was stirred for 2 h at 50° C. The reaction mixture was poured on 1N HCl/ice and EtOAc (500 mL) was added. Layers were separated the aqueous phase was extracted with EtOAc (2×250 mL). The combined organic layer was washed with water, dried over MgSO₄ and the solvent was removed under reduced pressure to give 9.5 g of crude product. Purification by column chromatography (Merck Silicagel 60, 0.040-0.063 mm, 230-400 mesh) using a gradient elution mixture (10:1 to 4:1 hexane:EtOAc) gave 9.5 g (100%) of the product, which solidifies upon standing at low temperature (8° C.).

¹H NMR (500 MHz, CDCl₃) δ=6.07 (s, 1H), 5.87 (s, 1H), 5.13 (bd, J=8.20 Hz, 1H), 5.06 (dt, J=3.15, 9.22 Hz, 1H), 1.90 (s, 3H), 1.73 (m, 1H), 1.47 (m, 1H), 1.43 (s, 9H), 1.32 (ddd, J=4.39, 9.77, 14.01 Hz, 1H), 1.00 (d, J=6.62 Hz, 3H), 0.90 (d, J=6.94 Hz, 3H); ¹³C NMR (125 MHz, CDCl₃) δ=201.6, 155.5, 142.3, 126.1, 79.6, 52.6, 43.2, 28.3, 25.0, 23.4, 21.7, 17.8.

### Example 3

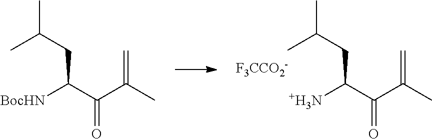 [SMILES: C=C(C)C(=O)[C@H](CC(C)C)NC(=O)OC(C)(C)C ; "F3CCO2-" ; C=C(C)C(=O)[C@@H]([NH3+])CC(C)C]

To Boc-Vinylketone (5 g, 19.6 mmol) in DCM (60 mL) at 0° C. was added TFA (7.56 mL, 98 mmol). The rxn was warmed to rt and stirred for 7 h. The solvent was removed and the TFA salt precipitated with ter-butyl methyl ether (TBME) and hexane at low temperature to give 3 g (61%) of the product after filtration and drying under vacuo.

¹H NMR (500 MHz, CDCl₃) δ=6.00 (d, J=1.26 Hz, 2H), 4.84 (dd, J=3.62, 9.93 Hz, 1H), 1.91 (s, 3H), 1.89 (m, 1H), 1.76 (ddd, J=4.77, 9.83, 14.75 Hz, 1H), 1.66 (ddd, J=3.67, 9.76, 14.66 Hz, 1H), 1.02 (d, J=6.54 Hz, 3H), 0.95 (d, J=6.62 Hz, 3H)

### Example 4

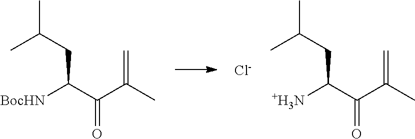 [SMILES: C=C(C)C(=O)[C@H](CC(C)C)NC(=O)OC(C)(C)C ; [Cl-] ; C=C(C)C(=O)[C@@H]([NH3+])CC(C)C]

Boc-Vinylketone (13.6 g, 53.3 mmol) was dissolved in ethanolic HCl (170 mL, 1.25M). The rxn was stirred for 18 h. The solvent was removed and the HCl salt crystallized with MTBE at low temperature to give 3.98 g (39%) of the product after filtration and drying under vacuo.

¹H NMR (500 MHz, CDCl₃) δ=8.69 (bs, 1H), 6.03 (s, 1H), 5.94 (d, J=1.57 Hz, 1H), 4.95 (m, 1H), 2.08 (m, 1H), 1.90 (s, 3H), 1.92 (ddd, J=4.67, 9.67, 14.56 Hz, 1H), 1.66 (ddd, J=3.71, 9.70, 14.57 Hz, 1H), 1.04 (d, J=6.30 Hz, 3H), 0.96 (d, J=6.30 Hz, 3H)

### Example 5

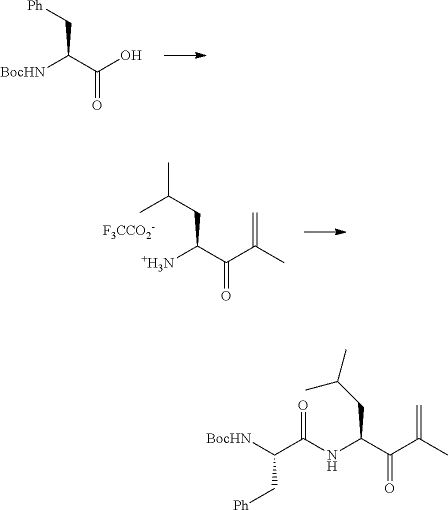 [SMILES: CC(C)(C)OC(=O)N[C@@H](Cc1ccccc1)C(=O)O ; "F3CCO2-" ; C=C(C)C(=O)[C@@H]([NH3+])CC(C)C ; C=C(C)C(=O)[C@H](CC(C)C)NC(=O)[C@H](Cc1ccccc1)NC(=O)OC(C)(C)C]

To a solution of Boc-Phe.OH (336 mg, 1.26 mmol), TBTU (497 mg, 1.54 mmol) and HOBt (210 mg, 1.54 mmol in THF (10 mL) at 0° C. was added a solution of TFA salt (350 mg, 1.26 mmol) in THF (3 mL) followed by DIPEA (660 μL, 3 mmol). The mixture was stirred at rt for 4 h, quenched by the addition of water and extracted with EtOAC. The combined organic layer was washed with water, dried over MgSO₄ and the solvent was removed under reduced pressure. Column chromatography (5:1 to 3:1 Hexane:EtOAc) gave 470 mg (93%) dipeptide.

### Example 6

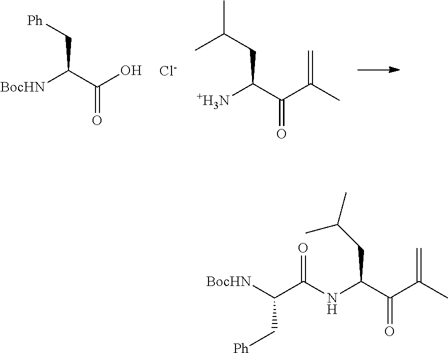 [SMILES: CC(C)(C)OC(=O)N[C@@H](Cc1ccccc1)C(=O)O ; [Cl-] ; C=C(C)C(=O)[C@@H]([NH3+])CC(C)C ; C=C(C)C(=O)[C@H](CC(C)C)NC(=O)[C@H](Cc1ccccc1)NC(=O)OC(C)(C)C]

To a solution of Boc-Phe-OH (8.8 g, 32.9 mmol), TBTU (13.1 g, 39.5 mmol) and HOBt (5.5 g, 39.5 mmol in THF (473 mL) at 0° C. was added DIPEA (17.1 mL, 98.7 mmol), and stirred for 10 min. Then a solution of HCl salt (6.3 g, 32.9 mmol) in THF (190 mL) was added. The mixture was stirred at rt for 2 h, quenched by the addition of brine and extracted with EtOAc. The combined organic layer was washed with water, dried over MgSO4 and the solvent was removed under reduced pressure. Column chromatography (5:1 to 3:1 Hexane:EtOAc) gave 17.8 g (100%) dipeptide.

¹H NMR (500 MHz, CDCl₃) δ=7.28-7.16 (m, 5H), 6.41 (d, J=8.19 Hz, 1H), 6.07 (s, 1H), 5.89 (s, 1H), 5.32 (m, 1H), 5.00 (bs, 1H), 4.34 (bs, 1H), 3.07 (dd, J=6.62, 13.87 Hz, 1H), 3.02 (dd, J=6.78, 13.71 Hz, 1H), 1.87 (s, 3H), 1.57 (m, 1H), 1.46 (ddd, J=3.94, 9.61, 13.71 Hz, 1H), 1.41 (s, 9H), 1.32 (ddd, J=4.33, 9.69, 13.95 Hz, 1H), 0.97 (d, J=6.62 Hz, 3H), 0.85 (d, J=6.62 Hz, 3H); ¹³C NMR (125 MHz, CDCl₃) δ=200.1, 170.7, 142.1, 128.6, 126.9, 126.5, 51.0, 43.1, 38.3, 28.2, 24.8, 23.3, 21.8, 17.7

### Example 7

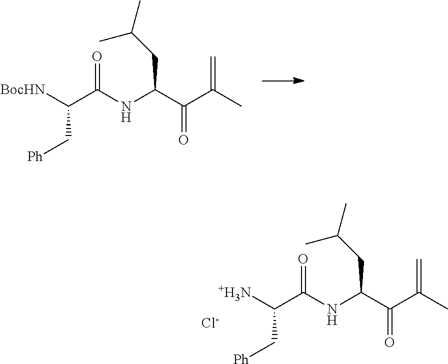 [SMILES: C=C(C)C(=O)[C@H](CC(C)C)NC(=O)[C@H](Cc1ccccc1)NC(=O)OC(C)(C)C ; [Cl-] ; C=C(C)C(=O)[C@H](CC(C)C)NC(=O)[C@@H]([NH3+])Cc1ccccc1]

The peptide (300 mg, 0.75 mmol) was dissolved in HCl (6 mL, 1.25M in EtOH) and stirred for 4 h. The solvent was removed under reduced pressure to give the HCl salt as white solid, which was directly used for the next step.

### Example 8

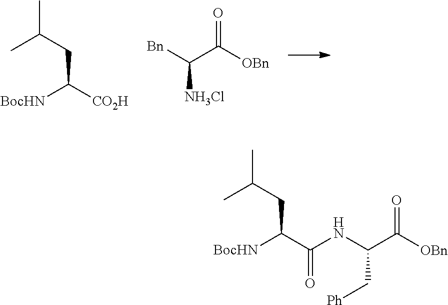

L-Phenylalanine benzyl ester hydrochloride (2.5 g, 8.6 mmol) was suspended in DCM (18 mL) and DMF (1.8 mL) under N₂ and the mixture was cooled to 0° C. with an ice bath. Triethylamine (1.3 mL, 9.46 mmol) was added followed by Boc-Leu-OH (2 g, 8.6 mmol) and HOBt (1.3 g, 9.46 mmol) and the white suspension was stirred for 5 min. Then EDC.HCl (1.85 g, 9.46 mmol) was added neat and a clear solution formed. The cooling bath was removed and after 2 h HPLC analysis showed full conversion. The reaction was quenched by pouring on aqueous HCl (54 mL; 0.5M). The layers were separated and the aqueous layer extracted two times with EtOAc. The combined organic layer was washed with brine and dried over MgSO₄. The solvent was removed under reduced pressure to give 4.8 g of crude material. Purification by column chromatography (48 g Merck Silicagel 60, 0.040-0.063 mm, 230-400 mesh) using a 1:1 heptane:EtOAc mixture as eluent gave 3.49 g (87%) of dipeptide as white crystalline powder.

¹H NMR (500 MHz, CDCl₃) δ=7.35 (m, 3H), 7.28 (m, 2H), 7.21 (m, 3H), 7.02 (m, 3H), 6.48 (d, J=7.90 Hz, 1H), 5.15 (d, J=11.95 Hz, 1H), 5.10 (d, J=12.05 Hz, 1H), 4.88 (dt, J=5.94, 7.72 Hz, 1H), 4.80 (bs, 1H), 4.08 (bs, 1H), 3.14 (dd, J=6.30, 13.55 Hz, 1H), 3.09 (dd, J=5.67, 13.88 Hz, 1H), 1.66-1.56 (m, 2H), 1.43 (s, 9H), 1.42 (m, 1H), 0.90 (d, J=6.30 Hz, 3H), 0.89 (d, J=6.30 Hz, 3H)

¹³C NMR (125 MHz, CDCl₃) δ=172.1, 171.0, 135.6, 135.0, 129.4, 128.6, 128.5, 127.1, 67.2, 53.1, 41.3, 37.9, 28.3, 24.7, 22.8, 21.9

### Example 9

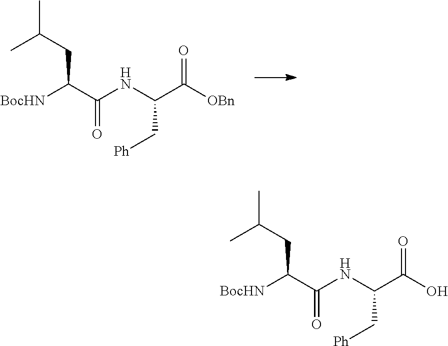 [SMILES: CC(C)C[C@H](NC(=O)OC(C)(C)C)C(=O)N[C@@H](Cc1ccccc1)C(=O)OCc1ccccc1 ; CC(C)C[C@H](NC(=O)OC(C)(C)C)C(=O)N[C@@H](Cc1ccccc1)C(=O)O]

Boc-Leu-Phe-OBn (600 mg, 1.28 mmol) was dissolved in EtOH (13 mL) and Pd/C (136 mg, 10%) was added and stirred under H₂ atmosphere for 1 h. The catalyst was filtered off over celite and the solvent as removed under reduced pressure to give 518 mg (100%) of the acid.

¹H NMR (500 MHz, d⁶-dmso) δ=12.08 (bs, 1H), 7.87 (d, J=7.88 Hz, 1H), 7.24 (m, 2H), 7.20 (m, 3H), 6.83 (d, J=8.51 Hz, 1H), 4.43 (dt, J=8.10, 5.22 HZ, 1H), 3.93 (dt, J=9.06, 5.52 Hz, 1H), 3.04 (dd, J=5.39, 13.84 Hz, 1H), 2.91 (dd, J=8.44, 13.94 Hz, 1H), 1.51 (m, 1H), 1.39 (s, 9H), 1.32 (m, 2H), 0.84 (d, J=6.62 Hz, 3H), 0.81 (d, J=6.62 Hz, 3H)

¹³C NMR (125 MHz, d⁶-dmso) δ=172.7, 172.2, 155.1, 137.3, 129.2, 128.1, 126.4, 78.0, 53.1, 52.8, 40.8, 36.7, 27.9, 24.1, 22.9, 21.5

### Example 10

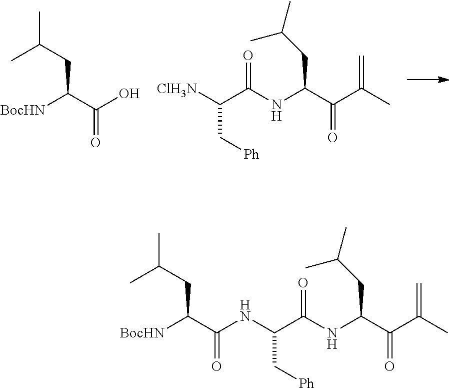

To a solution of the HCl salt (240 mg, 0.7 mmol) in DCM:DMF 10:1 (2.2 mL) was added Boc-Leu-OH (204 mg, 0.7 mmol), EDC.HCl (148 mg, 0.77 mmol) and HOBt (104 mg, 0.77 mmol). After complete dissolution Net3 (98 μL, 0.77 mmol) was added. The mixture was stirred for 16 h, diluted with water and extracted with DCM. The combined organic layer was washed with water, dried over MgSO₄ and the solvent removed under reduced pressure. Column chromatography (3%→10% MeOH in DCM) gave 250 mg (89%) of the product.

### Example 11

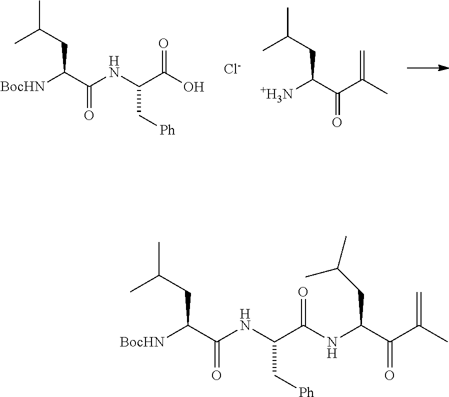 [SMILES: CC(C)C[C@H](NC(=O)OC(C)(C)C)C(=O)N[C@@H](Cc1ccccc1)C(=O)O ; [Cl-] ; C=C(C)C(=O)[C@@H]([NH3+])CC(C)C ; C=C(C)C(=O)[C@H](CC(C)C)NC(=O)[C@H](Cc1ccccc1)NC(=O)[C@H](CC(C)C)NC(=O)OC(C)(C)C]

To the acid (100 mg, 0.24 mmol) and the HCl salt (46 mg, 0.24 mmol) in DMF (2.5 mL) was added DIC (74 μL, 0.48 mmol) and HOBt (39 mg, 0.29 mmol). After 5 min DiPEA (42 μL, 0.24 mmol) was added and stirring continued for 18 h at rt. Water was added and the aqueous solution was extracted with EtOAc. The combined organic layer was washed with water, dried over MgSO₄ and the solvent removed under reduced pressure. Column chromatography (5% MeOH in DCM) gave 54 mg (44%) of the pure product.

¹H NMR (500 MHz, d⁶-dmso) δ=8.28 (d, J=7.88 Hz, 1H), 7.74 (d, J=8.19 Hz, 1H), 7.23-7.15 (m, 5H), 6.87 (d, J=8.51 Hz, 1H), 6.05 (s, 1H), 5.89 (d, J=1.26 Hz, 1H), 5.03 (dt, J=8.17, 6.04 Hz, 1H), 4.57 (dt, J=8.43, 5.20 Hz, 1H), 3.88 (dt, J=8.91, 5.20 Hz, 1H), 2.96 (dd, J=4.89, 14.02 Hz, 1H), 2.75 (dd, J=8.82, 13.87 Hz, 1H), 1.79 (s, 3H), 1.58 (m, 1H), 1.47 (m, 1H), 1.41 (m, 2H), 1.37 (s, 9H), 1.30-1.21 (m, 2H), 0.87 (d, J=6.30 Hz, 3H), 0.86 (d, J=6.62 Hz, 3H), 0.82 (d, J=6.62 Hz, 3H), 0.79 (d, J=6.55 Hz, 3H).

### Example 12

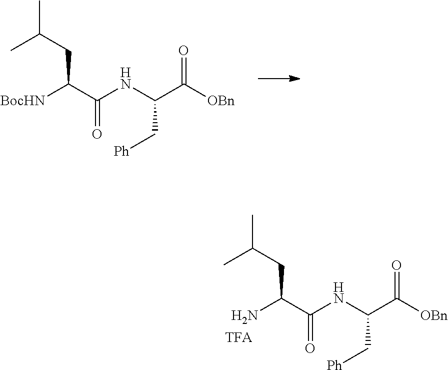 [SMILES: CC(C)C[C@H](NC(=O)OC(C)(C)C)C(=O)N[C@@H](Cc1ccccc1)C(=O)OCc1ccccc1 ; CC(C)C[C@H](N)C(=O)N[C@@H](Cc1ccccc1)C(=O)OCc1ccccc1]

Boc-Leu-Phe-OBn (3.4 g, 7.3 mmol) was dissolved in DCM (23 mL) and the solution was cooled to 0° C. TFA (7.7 mL, 99.3 mmol) was slowly added and the mixture was stirred at rt overnight. The solvent was removed under reduced pressure to give a yellow solid. The solid was with toluene and DCM. Then it was suspended in diethyl ether, filtered and washed with diethyl ether and dried under vacuum for 2 h at rt to give 3.26 g (96%) of white crystalline TFA salt.

¹H NMR (500 MHz, d⁶-dmso) δ=9.05 (d, J=7.25 Hz, 1H), 8.19 (bs, 3H), 7.34 (m, 3H), 7.30-7.22 (m, 7H), 5.09 (d, J=12.60 Hz, 1H), 5.06 (d, J=12.60 Hz, 1H), 4.62 (q, J=7.35 Hz, 1H), 3.78 (bt, J=6.77 Hz, 1H), 3.10 (dd, J=6.30, 14.15 Hz, 1H), 3.02 (dd, J=8.52, 14.03 Hz, 1H), 1.63 (m, 1H), 1.49 (t, J=7.25 Hz, 2H), 0.84 (d, J=6.60 Hz, 3H), 0.83 (d, J=6.75 Hz, 3H)

¹³C NMR (125 MHz, d⁶-dmso) δ=170.7, 169.4, 136.7, 135.5, 129.0, 128.4, 128.1, 127.9, 126.7, 66.2, 53.9, 50.5, 40.2, 36.4, 23.3, 22.7, 21.6

### Example 13

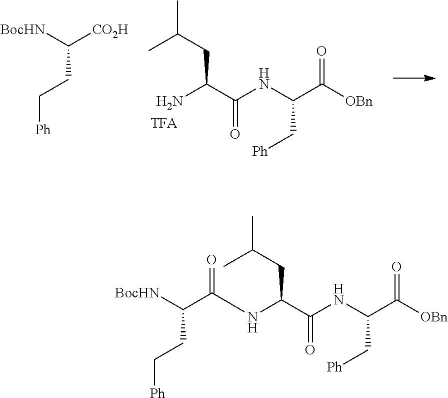 [SMILES: CC(C)(C)OC(=O)N[C@@H](CCc1ccccc1)C(=O)O ; CC(C)C[C@H](N)C(=O)N[C@@H](Cc1ccccc1)C(=O)OCc1ccccc1 ; CC(C)C[C@H](NC(=O)[C@H](CCc1ccccc1)NC(=O)OC(C)(C)C)C(=O)N[C@@H](Cc1ccccc1)C(=O)OCc1ccccc1]

To TFA-Phe-Leu-OBn (3.2 g, 6.6 mmol) in CH₃CN (38 mL) and DMF (5.7 mL) was added diisopropylethylamine (4.6 g, 26.4 mmol) followed by HOBt (1.0 g, 7.26 mmol), TBTU (2.4 g, 7.26 mmol) and Boc-Homophe-OH (1.84 g, 6.6 mmol). After 10 min a white precipitate formed and additional solvent, CH₃CN (38 mL) and DMF (5.7 mL), was added and the mixture was stirred overnight. The precipitate was filtered off, to give the first amount of product. The filtrated was treated with saturated aqueous NH₄Cl solution, a white precipitate formed, which dissolved upon the addition of water and EtOAc. Layers were separated; the organic layer was washed with water and brine, dried over MgSO₄ and the solvent removed. The combined white solids were washed with EtOAc and dried under vacuum to give 2.42 g (58%) of the product.

¹H NMR (500 MHz, CDCl₃) δ=7.34 (m, 3H), 7.30-7.25 (m, 4H), 7.21-7.14 (m, 6H), 7.00 (m, 2H), 6.48 (bd, J=6.99 Hz, 1H), 6.41 (bd, J=6.94 Hz, 1H), 5.15 (d, J=12.30 Hz, 1H), 5.08 (d, J=11.98 Hz, 1H), 4.96 (bd, J=6.94 Hz, 1H), 4.86 (dt, J=6.07, 7.72 Hz, 1H), 4.49 (m, 1H), 4.01 (m, 1H), 3.11 (dd, J=6.15, 13.85 Hz, 1H), 3.07 (dd, J=6.00, 13.85 Hz, 1H), 2.64 (t, J=7.90 Hz, 2H), 2.09 (m, 1H), 1.88 (m, 1H), 1.59 (m, 2H), 1.47 (m, 1H), 1.44 (s, 9H), 0.87 (d, J=6.65 Hz, 3H), 0.86 (d, J=6.90 Hz, 3H)

¹³C NMR (125 MHz, CDCl₃) δ=171.9, 171.3, 171.0, 135.5, 135.0, 129.3, 128.6, 128.5, 128.5, 128.4, 127.1, 126.2, 67.26, 53.21, 51.7, 40.9, 37.8, 33.5, 31.8, 28.3, 24.5, 22.9, 21.8

### Example 14

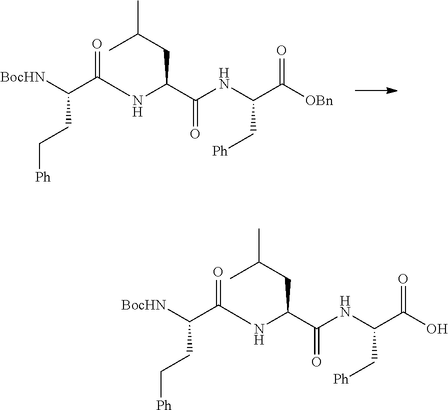 [SMILES: CC(C)C[C@H](NC(=O)[C@H](CCc1ccccc1)NC(=O)OC(C)(C)C)C(=O)N[C@@H](Cc1ccccc1)C(=O)OCc1ccccc1 ; CC(C)C[C@H](NC(=O)[C@H](CCc1ccccc1)NC(=O)OC(C)(C)C)C(=O)N[C@@H](Cc1ccccc1)C(=O)O]

The peptide (400 mg, 0.63 mmol) was dissolved in EtOH (7 mL) and Pd/C (68 mg, 10%) was added and stirred under H₂ atmosphere for 1 h. The catalyst was filtered off over celite and the solvent as removed under reduced pressure. The residue was stripped with EtOAc and Et₂O to give 370 mg (100%) of the acid.

¹H NMR (500 MHz, CDCl₃) δ=12.68 (bs, 1H), 8.15 (d, J=7.57 Hz, 1H), 7.73 (d, J=8.51 Hz, 1H), 7.27 (m, 2H), 7.17 (m, 7H), 7.10 (m, 1H), 7.04 (d, J=8.51 Hz, 1H), 4.41 (dt, J=8.09, 5.58 Hz, 1H), 4.36 (q, J=8.09 Hz, 1H), 3.90 (dt, J=8.30, 5.46 Hz, 1H), 3.02 (dd, J=5.36, 13.87 Hz, 1H), 2.89 (dd, J=8.67, 14.03 Hz, 1H), 2.58 (m, 1H), 2.47 (m, 1H), 1.80 (m, 1H), 1.74 (m, 1H), 1.59 (m, 1H), 1.42-1.36 (m, 2H), 1.39 (s, 9H), 0.86 (d, J=6.62 Hz, 3H), 0.82 (d, J=6.62 Hz, 3H)

¹³C NMR (125 MHz, d⁶-dmso) δ=172.6, 171.8, 171.5, 155.3, 141.6, 137.3, 128.9, 128.3, 128.0, 126.3, 125.7, 78.0, 54.0, 53.1, 50.6, 41.2, 36.5, 33.8, 31.6, 28.1, 23.9, 23.1, 21.6.

### Example 15

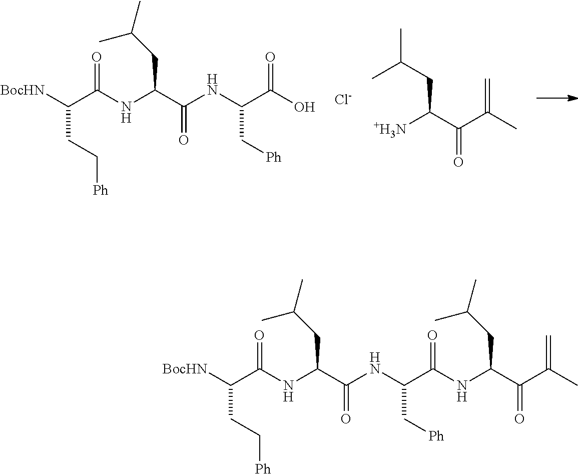 [SMILES: CC(C)C[C@H](NC(=O)[C@H](CCc1ccccc1)NC(=O)OC(C)(C)C)C(=O)N[C@@H](Cc1ccccc1)C(=O)O ; [Cl-] ; C=C(C)C(=O)[C@@H]([NH3+])CC(C)C ; C=C(C)C(=O)[C@H](CC(C)C)NC(=O)[C@H](Cc1ccccc1)NC(=O)[C@H](CC(C)C)NC(=O)[C@H](CCc1ccccc1)NC(=O)OC(C)(C)C]

To the acid (100 mg, 0.185 mmol) and the HCl salt (35 mg, 0.185 mmol) in DMF (2 mL) was added DIC (57 μL, 0.37 mmol) and HOBt (27 mg, 0.2 mmol). After 5 min DiPEA (32 μL, 0.185 mmol) was added and stirring continued for 18 h at rt. Water was added and the aqueous solution was extracted with EtOAc. The combined organic layer was washed with water, dried over MgSO₄ and the solvent removed under reduced pressure. Column chromatography (5% MeOH in DCM) gave 350 mg (99%) of the pure product.

¹H NMR (500 MHz, d⁶-dmso) δ=8.18 (d, J=8.19 Hz, 1H), 8.01 (d, J=8.19 Hz, 1H), 7.75 (d, J=8.19 Hz, 1H), 7.27 (m, 2H), 7.16 (m, 7H), 7.06 (m, 2H), 6.04 (s, 1H), 5.86 (d, J=1.26 Hz, 1H), 5.01 (dt, J=8.67, 5.04 Hz, 1H), 4.53 (dt, J=8.59, 5.20 Hz, 1H), 4.30 (dt, J=8.48, 5.73 Hz, 1H), 3.90 (dt, J=8.41, 5.24 Hz, 1H), 2.95 (dd, J=5.20, 14.03 Hz, 1H), 2.74 (dd, J=8.98, 14.03 Hz, 1H), 2.58 (m, 1H), 2.50 (m, 1H), 1.84-1.70 (m, 2H), 1.77 (s, 3H), 1.56 (m, 2H), 1.42-1.28 (m, 4H), 1.39 (s, 9H), 0.84 (m, 9H), 0.80 (d, J=6.62 Hz, 3H)

¹³C NMR (125 MHz, d⁶-dmso) δ=200.3, 171.6, 170.4, 155.3, 141.6, 141.5, 137.4, 129.0, 128.3, 128.2, 127.9, 126.1, 125.7, 125.6, 78.1, 54.0, 53.1, 50.7, 41.1, 37.3, 33.7, 31.6, 28.1, 24.2, 23.9, 23.1, 23.0, 21.6, 21.3, 17.6.

### Example 16

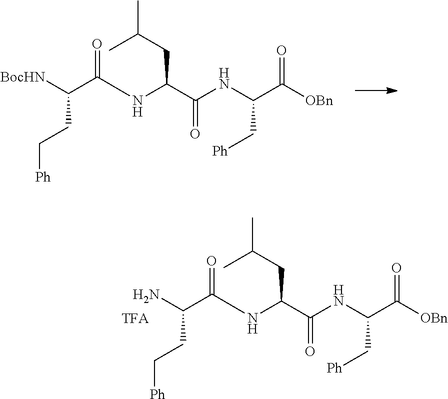 [SMILES: CC(C)C[C@H](NC(=O)[C@H](CCc1ccccc1)NC(=O)OC(C)(C)C)C(=O)N[C@@H](Cc1ccccc1)C(=O)OCc1ccccc1 ; CC(C)C[C@H](NC(=O)[C@@H](N)CCc1ccccc1)C(=O)N[C@@H](Cc1ccccc1)C(=O)OCc1ccccc1]

Boc-Homophe-Leu-Phe-OBn (2.4 g, 3.8 mmol) was dissolved in DCM (12 mL) and the solution was cooled to 0° C. TFA (3.5 mL, 45 mmol) was slowly added and the mixture was stirred at rt overnight. The solvent was removed under reduced pressure. The solid was stripped with toluene and DCM. Then it was dried under vacuum for 18 h at rt to give 2.5 g (100%) of white crystalline TFA salt.

¹H NMR (500 MHz, d⁶-dmso) δ=8.65 (d, J=7.55 Hz, 1H), 8.56 (d, J=8.50 Hz, 1H), 8.23 (bd, J=4.40 Hz, 3H), 7.35-7.24 (m, 7H), 7.22-7.13 (m, 7H), 7.07 (m, 1H), 5.04 (s, 2H), 4.57 (m, 1H), 4.43 (dt, J=6.62, 8.51 Hz, 1H), 3.87 (m, 1H), 3.06 (dd, J=5.87, 13.97 Hz, 1H), 2.96 (dd, J=8.93, 13.97 Hz, 1H), 2.55 (t, J=8.65 Hz, 2H), 1.92 (m, 2H), 1.60 (m, 1H), 1.40 (m, 2H), 0.87 (d, J=6.60 Hz, 3H), 0.84 (d, J=6.65 Hz, 3H)

¹³C NMR (125 MHz, d⁶-dmso) δ=171.8, 171.1, 140.8, 137.0, 135.6, 128.9, 128.4, 128.3, 128.1, 128.0, 127.9, 126.4, 126.1, 66.0, 53.4, 52.0, 50.8, 40.9, 36.4, 33.5, 30.3, 24.0, 23, 21.6.

### Example 17

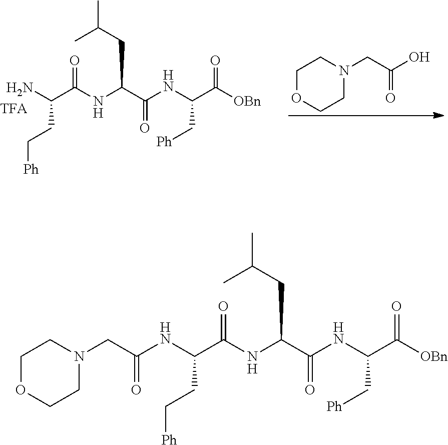 [SMILES: CC(C)C[C@H](NC(=O)[C@@H](N)CCc1ccccc1)C(=O)N[C@@H](Cc1ccccc1)C(=O)OCc1ccccc1 ; O=C(O)CN1CCOCC1 ; CC(C)C[C@H](NC(=O)[C@H](CCc1ccccc1)NC(=O)CN1CCOCC1)C(=O)N[C@@H](Cc1ccccc1)C(=O)OCc1ccccc1]

To TFA-Homophe-Leu-Phe-OBn (2.5 g, 3.9 mmol) in CH3CN (5 mL) and DMF (2.5 mL) at 0° C. was added DiPEA (3.4 mL, 19.5 mmol), EDC.HCl (0.92 g, 4.68 mmol) and HOBt (0.65 g, 4.68 mmol) followed by Morpholine acetic acid (0.68 g, 4.68 mmol). The reaction was stirred overnight, saturated, aqueous NH₄Cl solution was added and layers were separated. The aqueous layer was extracted with EtOAc, the combined organic layer was washed with water, dried over MgSO₄ and stripped with toluene and DCM to give 2.60 g (99%) of the product.

¹H NMR (500 MHz, d⁶-dmso) δ=8.47 (d, J=7.25 Hz, 1H), 8.05 (d, J=8.50 Hz, 1H), 7.88 (d, J=8.20 Hz, 1H), 7.33 (m, 3H), 7.26 (m, 4H), 7.19 (m, 5H), 7.14 (m, 3H), 5.04 (m, 2H), 4.53 (bq, J=7.38 Hz, 1H), 4.38 (m, 2H), 3.61 (bs, 4H), 3.05 (dd, J=6.08, 13.22 Hz, 1H), 3.00 (d, J=15.10 Hz, 1H), 2.99 (dd, J=5.38, 13.92 Hz, 1H), 2.94 (d, J=15.10 Hz, 1H), 2.44 (bs, 4H), 1.88 (m, 1H), 1.81 (m, 1H), 1.55 (m, 1H), 1.38 (m, 2H), 0.84 (d, J=6.60 Hz, 3H), 0.80 (d, J=6.30 Hz, 3H)

¹³C NMR (125 MHz, d⁶-dmso) δ=172.1, 171.1, 170.8, 168.8, 141.5, 136.9, 135.6, 128.9, 128.3, 123.3, 128.2, 128.1, 128.0, 127.8, 126.4, 125.8, 66.1, 66.0, 61.3, 53.5, 53.2, 51.7, 50.6, 40.9, 36.4, 34.5, 31.4, 24.0, 22.9, 21.6

### Example 18

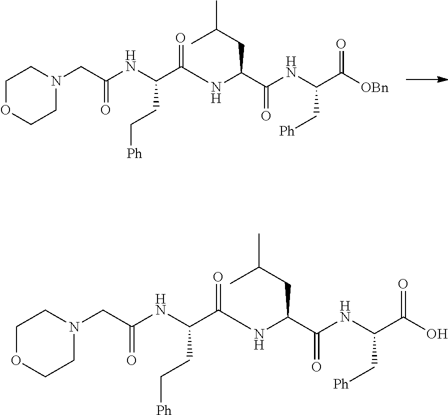 [SMILES: CC(C)C[C@H](NC(=O)[C@H](CCc1ccccc1)NC(=O)CN1CCOCC1)C(=O)N[C@@H](Cc1ccccc1)C(=O)OCc1ccccc1 ; CC(C)C[C@H](NC(=O)[C@H](CCc1ccccc1)NC(=O)CN1CCOCC1)C(=O)N[C@@H](Cc1ccccc1)C(=O)O]

Morph-Gly-Homophe-Leu-Phe-OBn (814 mg, 1.24 mmol) was suspended in EtOH (18 mL) and Pd/C (132 mg, 10%) was added and stirred under H₂ atmosphere for 2 h. The compound dissolved completely during the reaction. The catalyst was filtered off over a 0.2 μm PTFE filter and the solvent as removed under reduced pressure. The residue was stripped with EtOAc and Et₂O to give 711 mg (100%) of pale yellow crystals.

¹H NMR (500 MHz, d⁶-dmso) δ=8.16 (d, J=7.88 Hz, 1H), 8.05 (d, J=8.20 Hz, 1H), 7.88 (d, J=8.51 Hz, 1H), 7.27 (m, 2H), 7.19 (m, 5H), 7.15 (m, 2H), 7.10 (m, 1H), 4.41 (dt, J=8.14, 5.05 Hz, 1H), 4.36 (m, 2H), 3.60 (bs, 4H), 3.03 (dd, J=5.41, 13.79 Hz, 1H), 2.98 (d, J=15.13 Hz, 1H), 2.93 (d, J=15.05 Hz, 1H), 2.90 (dd, J=8.82, 13.87 Hz, 1H), 2.54-2.45 (m, 2H), 2.44 (bs, 4H), 1.88 (m, 1H), 1.80 (m, 1H), 1.57 (m, 1H), 1.41 (t, J=7.13 Hz, 2H), 0.87 (d, J=6.62 Hz, 3H), 0.82 (d, J=6.32 Hz, 3H)

¹³C NMR (125 MHz, d⁶-dmso) δ=172.7, 171.8, 170.8, 168.7, 141.5, 137.4, 128.9, 128.3, 128.2, 128.0, 126.3, 124.7, 66.1, 61.3, 53.2, 53.1, 51.7, 50.7, 40.8, 36.5, 34.4, 31.3, 24.0, 23.0, 21.6

### Example 19

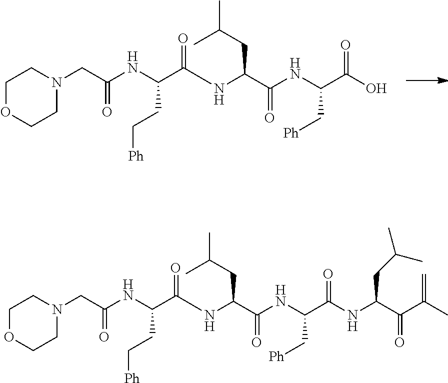 [SMILES: CC(C)C[C@H](NC(=O)[C@H](CCc1ccccc1)NC(=O)CN1CCOCC1)C(=O)N[C@@H](Cc1ccccc1)C(=O)O ; C=C(C)C(=O)[C@H](CC(C)C)NC(=O)[C@H](Cc1ccccc1)NC(=O)[C@H](CC(C)C)NC(=O)[C@H](CCc1ccccc1)NC(=O)CN1CCOCC1]

### TBTU/HOBt:

To the tetrapeptide (100 mg, 0.176 mmol), TBTU (70 mg, 0.211 mmol) and HOBt (29 mg, 0.211 mmol) in THF (2.5 mL) was added DIPEA (90 μL, 0.53 mmol) at 0° C. Then the HCl salt (34 mg, 0.176 mmol) in THF (1 mL) was added. After stirring the mixture for 2 h at rt brine was added and extracted with EtOAc. The combined organic layer was washed with water, dried over MgSO₄ and the solvent removed under reduced pressure to give 124 mg (100%) of crude product.

### DIC/HOBt:

To the tetrapeptide (100 mg, 0.176 mmol) and the HCl salt (34 mg, 0.176 mmol) in DMF (2.5 mL) was added DIC (56 μL, 0.35 mmol) and HOBt (25 mg, 0.176 mmol). After stirring the mixture for 18 h at rt water was added and extracted with EtOAc. The combined organic layer was washed with water, dried over MgSO₄ and the solvent removed under reduced pressure to give 121 mg (98%) of crude product.

### DIC/HOBt/CuCl₂:

A solution of the tetrapeptide (100 mg, 0.176 mmol) and the HCl salt (34 mg, 0.176 mmol) in DMF (0.5 mL) was added to CuCl₂ (24 mg, 0.176 mmol) dissolved in DMF (2 mL). DIC (56 μL, 0.35 mmol) and HOBt (25 mg, 0.176 mmol) were added. After stirring the mixture for 18 h at rt EtOAc was added and the organic layer was washed with NH₃ (7% in water), 1M HCl, water and brine. The organic layer was dried over MgSO₄ and the solvent removed under reduced pressure to give 94 mg (76%) of crude product.

### DIC/Oxyma:

To the tetrapeptide (100 mg, 0.176 mmol) and the HCl salt (34 mg, 0.176 mmol) in DMF (2.5 mL) was added DIC (56 μL, 0.35 mmol) and Oxyma (26 mg, 0.176 mmol). After stirring the mixture for 18 h at rt water was added and extracted with EtOAc. The combined organic layer was washed with water and concentrated to dryness. The residue was dissolved in MeTHF and washed with 1M NaOH, water and brine; dried over MgSO₄ and the solvent removed under reduced pressure to give 110 mg (89%) of crude product.

### DIC/Oxyma/CuCl₂:

The tetrapeptide (100 mg, 0.176 mmol) and the HCl salt (34 mg, 0.176 mmol) was added to a solution of CuCl₂ (24 mg, 0.176 mmol) dissolved in DMF (2.5 mL). DIC (56 μL, 0.35 mmol) and Oxyma (26 mg, 0.176 mmol) were added. After stirring the mixture for 18 h at rt EtOAc was added and the organic layer was washed with NH₃ (7% in water), 1M HCl, water and brine. The organic layer was dried over MgSO₄ and the solvent removed under reduced pressure to give 81 mg (65%) of crude product.

### EDC/HOBt/CuCl₂:

The tetrapeptide (500 mg, 0.88 mmol) and the HCl salt (169 mg, 0.88 mmol) were added to a solution of CuCl₂ (118 mg, 0.88 mmol) dissolved in DMF (12.5 mL). EDC.HCl (344 mg, 1.76 mmol) and HOBt (122 mg, 0.88 mmol) were added. After stirring the mixture for 18 h at rt EtOAc was added and the organic layer was washed with NH₃ (7% in water), 1M HCl, water and brine. The organic layer was dried over MgSO₄ and the solvent removed under reduced pressure to give 398 mg (64%) of crude product.

### ClCO₂iBu/NMM:

To the tetrapeptide (100 mg, 0.176 mmol) in DCM (2 mL) at 0° C. was added NMM (59 μL, 0.53 mmol) and ClCO₂iBu (26 μL, 0.19 mmol). After stirring the mixture for 30 min at rt the HCl salt (34 mg, 0.176 mmol) in DCM (0.5 mL) was added. After 2 h at rt water was added and extracted with EtOAc. The combined organic layer was washed with water, dried over MgSO₄ to give 98 mg (79%) of crude product.

¹H NMR (500 MHz, d⁶-dmso) δ=8.18 (d, J=8.19 Hz, 1H), 8.04 (d, J=8.20 Hz, 1H), 8.02 (d, J=8.51 Hz, 1H), 7.87 (d, J=8.19 Hz, 1H), 7.25 (m, 2H), 7.15 (m, 8H), 7.07 (m, 1H), 6.03 (s, 1H), 5.87 (s, 1H), 5.02 (dt, J=8.67, 5.04 Hz, 1H), 4.53 (dt, J=8.51, 5.04 Hz, 1H), 4.36 (dt, J=8.29, 5.16 Hz, 1H), 4.29 (q, J=7.78 Hz, 1H), 3.60 (bs, 4H), 2.96 (m, 3H), 2.76 (dd, J=8.99, 14.02 Hz, 1H), 2.50 (m, 2H), 2.43 (bs, 4H), 1.89 (m, 1H), 1.80 (m, 1H), 1.78 (s, 3H), 1.55 (m, 2H), 1.38 (m, 4H), 0.85 (d, J=6.30 Hz, 3H), 0.84 (d, J=6.62 Hz, 6H), 0.80 (d, J=6.62 Hz, 3H)

¹³C NMR (125 MHz, d⁶-dmso) δ=200.3, 171.5, 170.9, 170.5, 170.4, 168.8, 141.7, 141.5, 137.5, 129.1, 129.0, 128.2, 127.9, 127.8, 126.1, 125.7, 125.6, 66.1, 61.3, 53.2, 53.1, 51.7, 50.9, 50.7, 40.8, 40.0, 38.2, 37.3, 34.3, 31.4, 24.2, 24.0, 23.1, 23.0, 21.6, 21.3, 17.6.

### Example 20

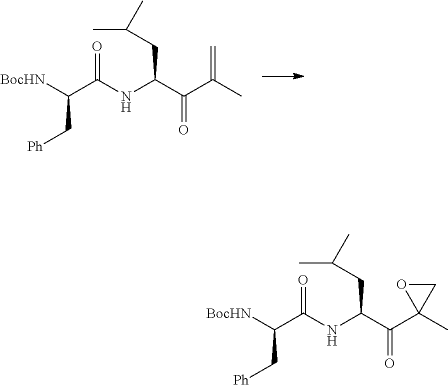 [SMILES: C=C(C)C(=O)[C@H](CC(C)C)NC(=O)[C@@H](Cc1ccccc1)NC(=O)OC(C)(C)C ; CC(C)C[C@H](NC(=O)[C@@H](Cc1ccccc1)NC(=O)OC(C)(C)C)C(=O)C1(C)CO1]

H₂O₂ in MeOH and NaOH

1.5 g of the vinyl ketone (3.7 mmol) was dissolved in 30 ml MeOH and the solution was cooled to 0° C. Subsequently 900 μl 35% H₂O₂ solution in water (2.7 eq) in 2 ml MeOH and 105 mg potassium hydroxide (0.5 eq) dissolved in 5 ml MeOH was added drop wise. The mixture was stirred over night and the temperature rised to room temperature. After 96% conversion the mixture was hydrolsed with 50 ml water and the product was extracted with 50 ml dichloromethane. The aqueous phase was reextracted with 50 ml dichloromethane and the combined organic phases were washed with 50 ml 1M sodium thiosulfate solution and brine. After evaporation to dryness 1.5 g (97%) of a white solid was isolated which contain an approx. 9/1 ratio of two diastereomers of the desired product.

m-Chloroperbenzoic acid

200 mg of the vinyl ketone was dissolved in 5 ml dichloro methane. To the solution 102 mg mCPBA (1.2 eq) was added. The mixture was stirred over night to achieve 59% conversion (HPLC). Additional 102 mg mCPBA was added and the mixture was stirred for additional 19 h to achieve 69% conversion. The mixture was hydrolysed with 10 ml water; the organic phase was separated and evaporated to dryness. 220 mg a white solid was isolated, containing 23 area % of starting material (HPLC) and 1/1 mixture of two diastereomers of the desired product.

### Ca(OCl)₂

200 mg of the vinyl ketone was dissolved in 2 ml N-Methylpyrrolidone and cooled to 0° C. To the solution a solution of 215 mg (4 eq) calcium hypochlorite in 0.5 ml water and 4 ml N-Methylpyrrolidone was added drop wise at 0° C. The reaction mixture was stirred overnight and the temperature increased to 20° C. After 55% conversion 5 ml of 1M sodium thiosulfate solution was added to the mixture. Afterwards the mixture was extracted twice with 10 ml of Hexan/MTBE mixture (8/2). The combined organic phase was separated and washed three times with 10 ml water. The organic solvent was removed to dryness and 150 mg of a white solid was isolated, containing 51 area % (HPLC) starting material and a 3/1 mixture of two diastereomers of the desired product.

¹H NMR (300 MHz, CDCl₃) δ=7.31-7.19 (m, 5H), 6.16 (d, J=7.2 Hz, 1H), 4.57 (m, 1H), 4.30 (m, 1H), 3.27 (d, J=4.1, 1H), 3.05 (m, J=6.62, 2H), 2.88 (d, J=5.0, 1H), 1.49 (s, 3H), 1.57-1.46 (m, 3H), 1.41 (s, 9H), 0.92 (d, J=6.1 Hz, 3H), 0.87 (d, J=6.2 Hz, 3H)

### Example 21

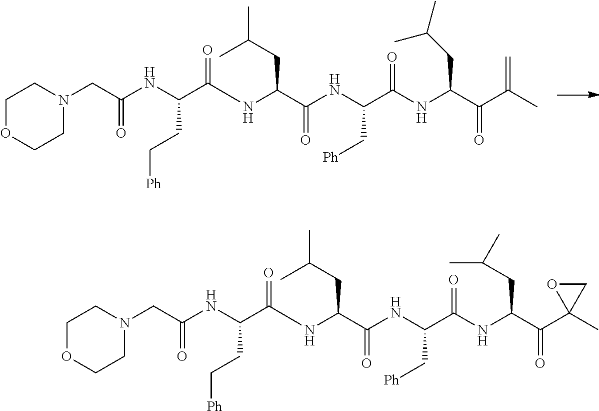 [SMILES: C=C(C)C(=O)[C@H](CC(C)C)NC(=O)[C@H](Cc1ccccc1)NC(=O)[C@H](CC(C)C)NC(=O)[C@H](CCc1ccccc1)NC(=O)CN1CCOCC1 ; CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)C1(C)CO1]

0.5 g of the vinyl ketone was dissolved in 10 ml MeOH and the solution was cooled to 0° C. Subsequently 160 μl 35% H₂O₂ solution in water (2.7 eq) in 1 ml MeOH and 20 mg potassium hydroxide (0.5 eq) dissolved in 1 ml MeOH was added drop wise. The mixture was stirred over night and the temperature rose to room temperature. 60% conversion and a 5/1 mixture of two diastereomers of the desired product was observed.

1. A method for preparing a compound of formula (I) 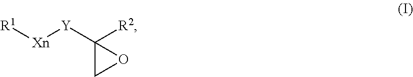
or a pharmaceutically acceptable salt or solvate thereof, the method comprising:
(i) Providing a compound of formula (II) 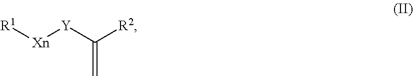
(ii) Epoxidizing the compound of formula (II) under conditions to obtain the compound of formula (I) or a pharmaceutically acceptable salt, hydrate or solvate thereof,
wherein
n is an integer between 1 and 1.000; preferably 1, 2, 3, 4, 5, 6, 7, 8, 9 or 10,
R¹ is R³-A-Q,
Q is selected from C(O), C(S), C—OH, C—SH, SO₂; or Q is absent,
A is selected from O, NH, C₁₋₇-alkyl, C₁₋₇-alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S; or A is absent,
R³ is selected from PG (protecting group), (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, the heteroatom is selected from O, N and/or S, wherein in case of nitrogen it can be provided as N-Oxide,
PG¹ is a nitrogen-protecting group, preferably selected from carbamates, amides, N-alkyl and N-aryl amines, quaternary ammonium salts, N-sulfonyl derivatives, halogen,
R² is selected from hydrogen, C₁₋₆-alkyl,
Xn is a chain of amino acids of n units X, each unit X is NR⁴—CHR⁵—C(O), R⁴ and R⁵ of adjacent units X are independently equal or different, preferably R⁵ between adjacent units is different,
Y is NR⁶—CHR⁷—C(O),
each R⁴ and R⁶ are independently selected from hydrogen, C₁₋₆-alkyl,
each R⁵ and R⁷ are independently selected from hydrogen, C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
optionally
(iii) replacing the PG by another group as defined for R³, provided that R³ is selected from PG.

2. The method according to claim 1, wherein the compound of formula (II) is prepared by a process comprising the steps:
Reacting a compound of formula (III) 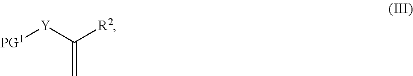
or a compound of formula (IV) 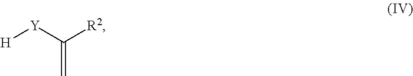
or a salt of a compound of formula (IV) with a compound of formula (V)
R¹-Xn-OH (V),
under conditions to obtain the compound of formula (II), 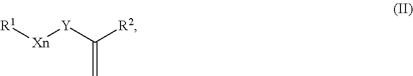
wherein
n, PG, X, Y, R¹ and R² are as defined above,
PG¹ is a nitrogen-protecting group, preferably selected from carbamates, amides, N-alkyl and N-aryl amines, quaternary ammonium salts, N-sulfonyl derivatives, halogen.

3. The method according to claim 1, wherein the compound of formula (II) is prepared by a process comprising the steps:
Reacting a compound of formula (III) 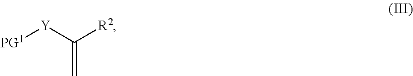
or a compound of formula (IV) 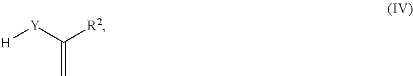
or a salt of a compound of formula (IV) with a compound of formula (VI)
PG²-X(n-m)-OH (VI),
under conditions to obtain the compound of formula (VII), 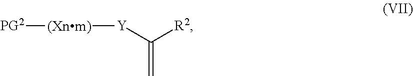
and subsequently coupling of m units X sequence wise, or of a sequence of m units X, according to the sequence Xn, with the compound of formula (VII) to obtain the compound of formula (II), 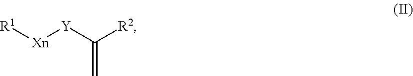
wherein
PG, X, Y, R¹ and R² are as defined in claim 1,
PG¹ is a nitrogen-protecting group, preferably selected from carbamates, amides, N-alkyl and N-aryl amines, quaternary ammonium salts, N-sulfonyl derivatives, halogen,
PG² is a nitrogen-protecting group, preferably selected from carbamates, amides, N-alkyl and N-aryl amines, quaternary ammonium salts, N-sulfonyl derivatives, halogen,
n is an integer between 2 and 1.000; preferably 2, 3, 4, 5, 6, 7, 8, 9 or 10,
m is an integer between 1 and n−1,
X(n-m) is a chain of amino acid of n units X of sequence Xn, lacking an amino (N—) terminal sequence of m units X of the sequence Xn.

4. The method according to claim 1, wherein
Xn is selected from the group consisting of:

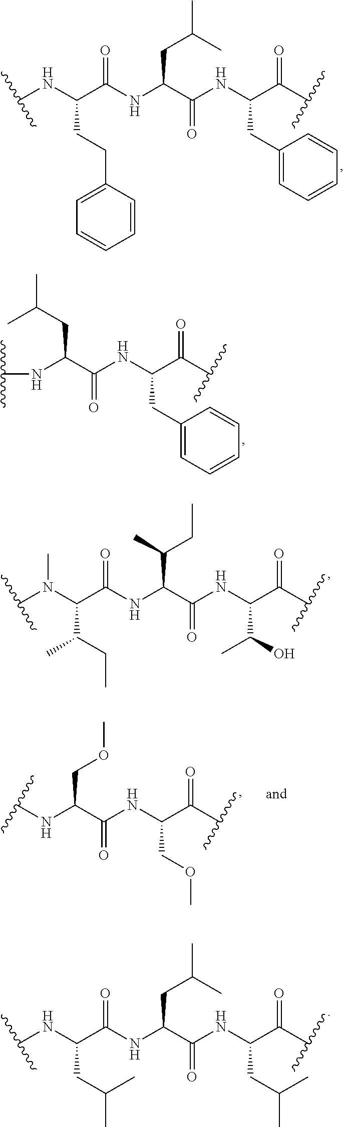

5. The method according to claim 3, wherein the preparation of the compound of formula (II) comprises the steps:
Reacting a compound of formula (III) 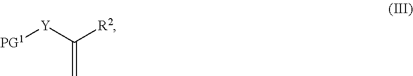
or a compound of formula (IV) 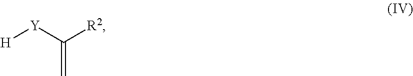
or a salt of a compound of formula (IV) with a compound of formula (VIII) 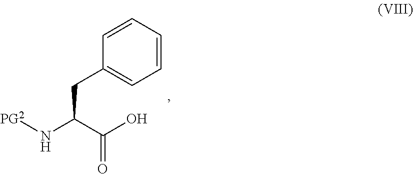
under conditions to obtain the compound of formula (IX), 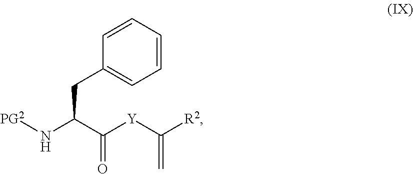
Reacting the compound of formula (IX) with a compound of formula (X) 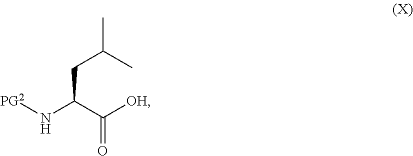
under conditions to obtain the compound of formula (XI), 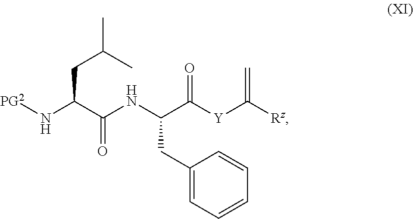
Reacting the compound of formula (XI) with a compound of formula (XII) 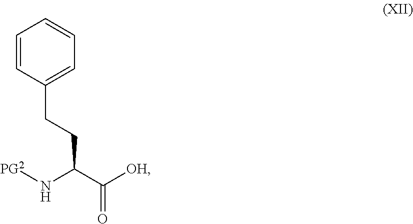
or with a compound of formula (XIII) 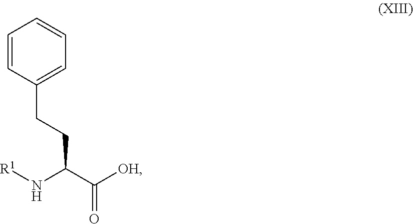
under conditions to obtain the compound of formula (XIV) or (XV), 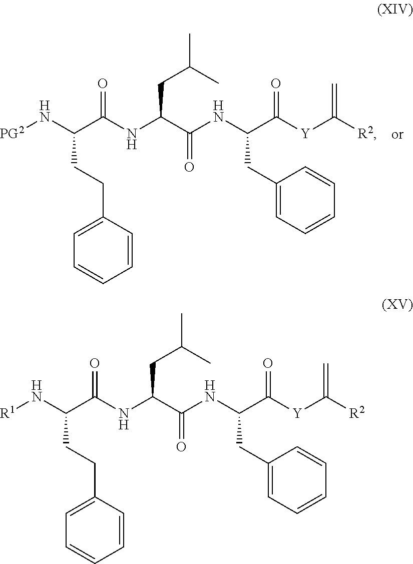
optionally, subsequently replacing the structural component PG² of the compound of formula (XIV) by the structural component R¹,
wherein
Y, R¹, R², PG, PG¹ and PG² are as defined above.

6. The method according to claim 2, wherein the compound of formula (V) is selected from the group consisting of:

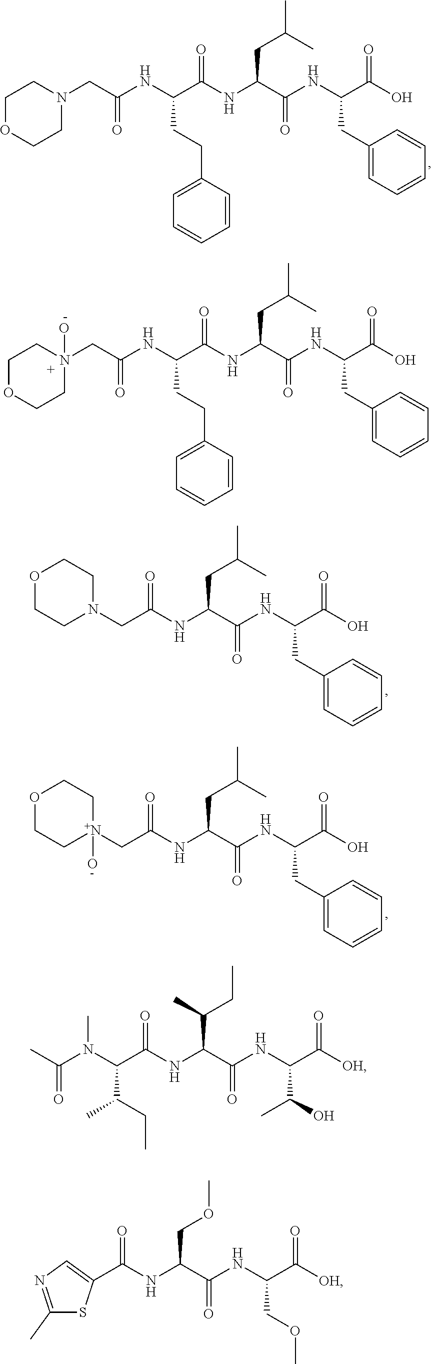

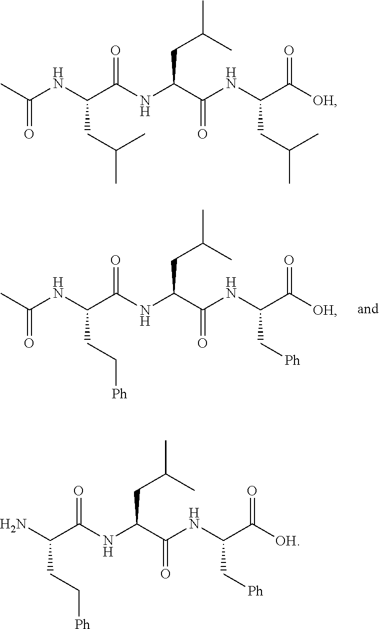

7. The method according to claim 1, wherein the compound of formula (I) is selected from the group consisting of:

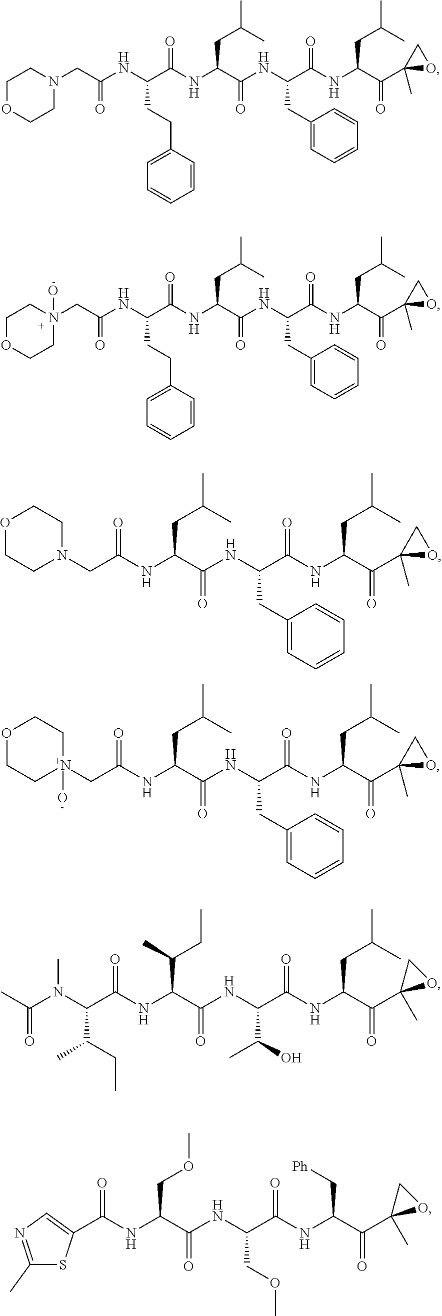

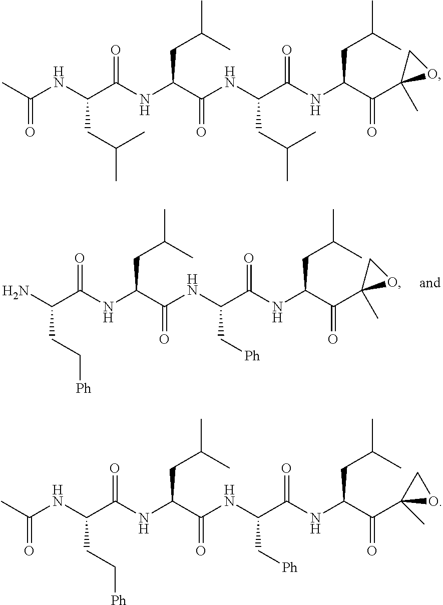 [SMILES: CC(=O)N[C@@H](CC(C)C)C(=O)N[C@@H](CC(C)C)C(=O)N[C@@H](CC(C)C)C(=O)N[C@@H](CC(C)C)C(=O)[C@@]1(C)CO1 ; CC(C)C[C@H](NC(=O)[C@@H](N)CCc1ccccc1)C(=O)N[C@@H](Cc1ccccc1)C(=O)N[C@@H](CC(C)C)C(=O)[C@@]1(C)CO1 ; CC(=O)N[C@@H](CCc1ccccc1)C(=O)N[C@@H](CC(C)C)C(=O)N[C@@H](Cc1ccccc1)C(=O)N[C@@H](CC(C)C)C(=O)[C@@]1(C)CO1]

8. The method according to claim 1, wherein the compound of formula (III) 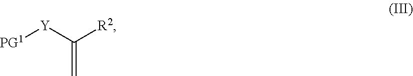
or the compound of formula (IV) 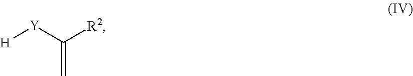
or a salt thereof
is prepared by a process comprising the steps of:
(a) Providing a compound of formula (XVI)
PG¹-Y-PG³ (XVI),
(b) Reacting the compound of formula (XVI) with a compound of formula (XVIa) 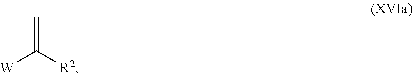
under conditions to obtain the compound of formula (III)
(c) Optionally removing of PG¹ of the compound of formula (III) under conditions to obtain the compound of formula (IV) or a salt thereof,
wherein
PG¹, Y and R² are as defined above,
PG³ is a Carboxyl-protection group, preferably selected from pyrrolidine, morpholine,
W is Li, MgCl, MgBr or MgI.

9. The method according to claim 8, wherein the salt of the compound of formula (IV) is formed by a cation which is 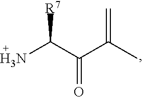
and an anion, the anion is preferably selected from F₃CCO₂⁻, nitrate, sulfate, halogen, such as chloride, bromide, iodide,
wherein
R₇ is selected from hydrogen, methyl, isopropyl, sec-butyl, isobutyl, homobenzyl or benzyl.

10. The method according to claim 8, wherein the compound of formula (III) is 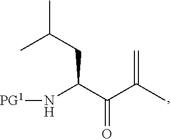
the compound of formula (IV) is 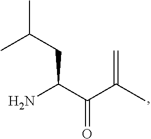
and/or the salt of the compound of formula (IV) is formed by a cation which is 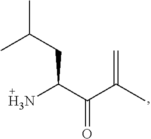
and an anion, the anion is preferably selected from F₃CCO₂⁻, nitrate, sulfate, halogen, such as chloride, bromide, iodide.

11. The method according to claim 5, wherein reactions to obtain at least one of the compounds of formula (II), (VII), (IX), (XI), (XIV) and (XV) are performed in the presence of at least one Lewis acid, preferably CuCl₂.

12. A salt of the compound of formula (IV), 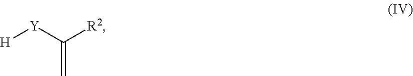
which is formed by a cation and an anion, the anion is preferably selected from F₃CCO₂⁻, nitrate, sulfate, halogen, such as chloride, bromide, iodide,
wherein
Y is NR⁶—CHR⁷—C(O),
R⁶ is selected from hydrogen, C₁₋₆-alkyl,
R⁷ is selected from C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
R² is selected from hydrogen, methyl, ethyl, n-propyl, isopropyl, isobutyl, sec-butyl, t-butyl, n-pentyl, isopentyl, t-pentyl, neo-pentyl, sec-pentyl, 3-pentyl, n-hexyl, sec-hexyl, t-hexyl, iso-hexyl.

13. A compound of formula (II) 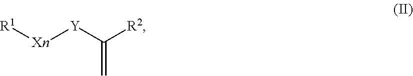
wherein
n is 2, 3, 4, 5, 6, 7, 8, 9 or 10,
R¹ is R³-A-Q,
Q is selected from C(O), C(S), C—OH, C—SH, SO₂; or Q is absent,
A is selected from O, NH, C₁₋₇-alkyl, C₁₋₇-alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S; or A is absent,
R³ is selected from PG (protecting group), hydrogen, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, the heteroatom is selected from O, N and/or S, wherein in case of nitrogen it can be provided as N-Oxide,
PG is a nitrogen-protecting group, preferably selected from carbamates, amides, N-alkyl and N-aryl amines, quaternary ammonium salts, N-sulfonyl derivatives, halogen,
R² is linear or branched C₁₋₆-alkyl,
Xn is a chain of amino acids of n units X, each unit X is NR⁴—CHR⁵—C(O), R⁴ and R⁵ of adjacent units X are independently equal or different, preferably R⁵ between adjacent units is different,
Y is NR⁶—CHR⁷—C(O),
each R⁴ and R⁶ are independently selected from hydrogen, C₁₋₆-alkyl,
R⁵ is selected from hydrogen, C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
R⁷ is selected from hydrogen, C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S.

14. The compound according to claim 13, wherein
Xn is a sequence selected from the group consisting of: 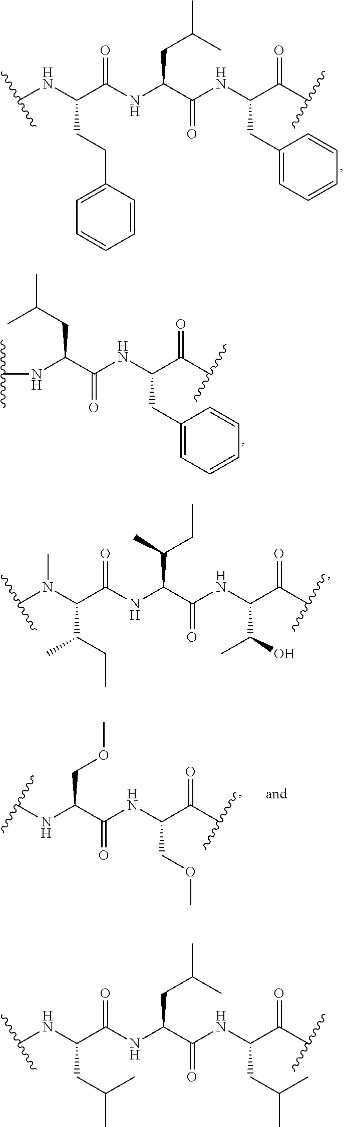
or X is

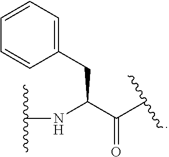 [SMILES: *N[C@@H](Cc1ccccc1)C(*)=O]

15. The compound of formula (II) according to claim 13, wherein the compound of formula (II) is selected from the group consisting of:

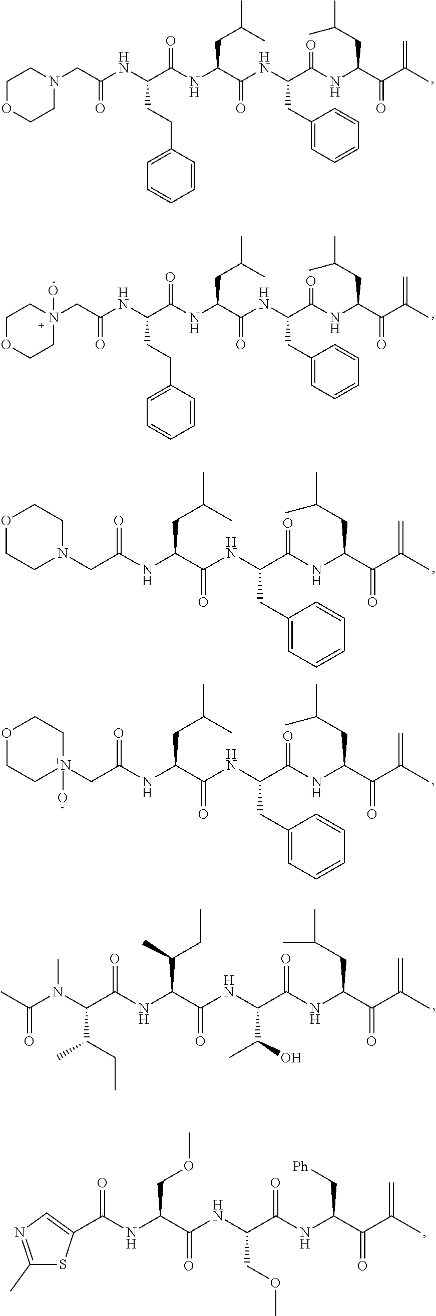

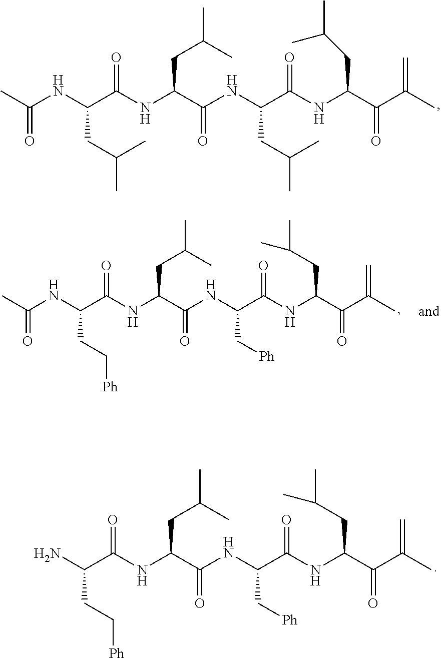

16. A pharmaceutical composition, comprising a compound of formula (I), wherein the composition is free or substantially free of a compound of formula (XVII) 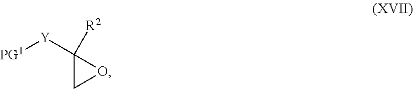
and/or formula (XVIII) 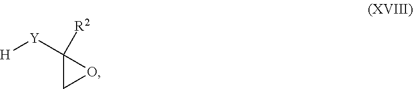
or a salt of the compound of formula (XVIII),
Y is NR⁶—CHR⁷—C(O),
R⁶ is selected from hydrogen, C₁₋₆-alkyl,
R⁷ is selected from C₁₋₂₀alkyl, C₁₋₂₀alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S,
R² is selected from hydrogen, methyl, ethyl, n-propyl, isopropyl, isobutyl, sec-butyl, t-butyl, n-pentyl, isopentyl, t-pentyl, neo-pentyl, sec-pentyl, 3-pentyl, n-hexyl, sec-hexyl, t-hexyl, iso-hexyl,
R¹ is R³-A-Q,
Q is selected from C(O), C(S), C—OH, C—SH, SO₂; or Q is absent,
A is selected from O, NH, C₁₋₇-alkyl, C₁₋₇-alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, or is substituted with one or more of unbranched or branched C₁₋₂₀-(hetero)alkyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally further substituted, the heteroatom is selected from O, N and/or S; or A is absent,
R³ is selected from PG (protecting group), (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkynyl, any of which is optionally substituted with one or more of a group selected from oxo, oxy, hydroxy, carboxy, alkoxy, alkoxycarbonyl, carbamoyl, amino, imido, imino, thioyl, sulfonyl, sulfinyl, sulfo, sulfanyl, disulfanyl, the heteroatom is selected from O, N and/or S, wherein in case of nitrogen it can be provided as N-Oxide,
PG¹ is a nitrogen-protecting group, preferably selected from carbamates, amides, N-alkyl and N-aryl amines, quaternary ammonium salts, N-sulfonyl derivatives, halogen,
wherein the composition further contains between
0.005% (w/w) and 5% (w/w) or 0.01% (w/w) and 1% (w/w) of the compound of formula (II) 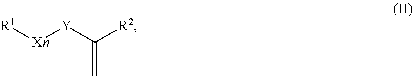
and/or
0.005% (w/w) and 5% (w/w) or 0.01% (w/w) and 1% (w/w) of the compound of formula (IV) 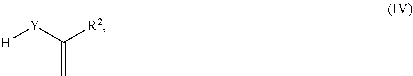
and
wherein the structural components Y and R² between the compounds of formulae (I), (II), (IV), (XVII) and (XVIII) are identical.
